(19) United States
(12) Patent Application Publication
(10) Pub. No.: US 2024/0075058 A1
(43) Pub. Date: Mar. 7, 2024

(54) METHOD AND USE OF COMPOSITIONS COMPRISING LIGNOSULFONATE FOR PATHOGENIC ATTENUATION

(71) Applicant: Attenubiotics Inc., Calgary (CA)

(72) Inventors: Ralph Kevin Walker, Calgary (CA); Sudha Bhavanam, Edmonton (CA)

(21) Appl. No.: 18/370,836

(22) Filed: Sep. 20, 2023

Related U.S. Application Data
(63) Continuation-in-part of application No. 16/523,901, filed on Jul. 26, 2019.
(60) Provisional application No. 62/703,755, filed on Jul. 26, 2018.

(51) Int. Cl. A61K 31/795; A23K 20/111; A23K 50/30; A61K 9/00; A61P 3/10; A61P 31/12; A61P 37/06

(52) U.S. Cl. CPC A61K 31/795; A23K 20/111; A23K 50/30; A61K 9/0053; A61P 3/10; A61P 31/12; A61P 37/06

(57) ABSTRACT

The use of a composition which comprises lignosulfonate for the prevention and treatment of pathogenic and medical disorders in humans and animals. In some embodiments the composition is substantially free of elemental sulphur. In some embodiments the lignosulfonate is radically polymerized. In some embodiments the composition is formulated as an animal feed additive or supplement. The disclosure also encompasses a method of preventing or treating a pathogenic or medical disorder in a human or animal subject by administering the composition to the subject in an effective dose to attenuate the pathogenic effect of a pathogen or other biological agent, thereby enabling the subject to mount an effective immune response to the pathogen or other biological agent. The composition can be used in the prevention or treatment of a wide range of pathogenic or medical disorders including disorders caused by microbial pathogens; disorders caused by viral pathogens; disorders caused by prions; disorders caused by protists; disorders caused by fungi; disorders caused by parasites; lung and airway disorders; bone, joint and muscle disorders; digestive disorders; hormonal disorders; cancer; auto immune disorders; neurodegenerative disorders; skin disorders; and sexual and reproductive disorders. In one particular embodiment the composition is formulated for treatment of Type 1 diabetes.

### RELATED APPLICATIONS

This application is a continuation-in-part of U.S. patent application Ser. No. 16/523,901 entitled METHOD AND USE OF COMPOSITIONS COMPRISING LIGNOSULFONATE AND SUBSTANTIALLY FREE OF ELEMENTAL SULPHUR FOR PATHOGENIC ATTENUATION filed 26 Jul. 2019 which claims the benefit of U.S. provisional patent application No. 62/703,755 filed 26 Jul. 2018. Each of the foregoing applications is hereby incorporated herein by reference in its entirety for all purposes.

### TECHNICAL FIELD

This application relates to the use of compositions comprising lignosulfonate for prevention and treatment of pathogenic and medical disorders in humans and animals.

### BACKGROUND

Many medical disorders are caused by pathogenic agents such as bacteria or viruses. Conventional treatment of such disorders is focused on killing or removing the pathogen, such as by administering antibiotics or anti-viral drugs. However, such treatment is often not effective. For example, bacteria tend to develop resistance to antibiotics over time, requiring the development of new drugs or drug combinations or other therapies.

Compositions intended to attenuate the effect of pathogenic agents are known in the prior art. For example, United States Patent Application Publication No. US 2015/0132390 A1, which is hereby incorporated by reference in its entirety, describes a pharmaceutical preparation comprising as an active ingredient micron-sized sulphur particles. The preparation may also include sodium lignin sulphate (sometimes referred to as sodium lignosulfonate). As described in the '390 publication, the purpose of the sulphur particles is not to kill a target pathogen but rather to re-establish a healthy equilibrium or homeostasis from a pathological imbalance or disorder. According to the theory of the '390 disclosure, the compound is believed to act by depriving the pathogen of oxygen with available sulphur atoms that are oxidized. This results in a “pathogenic attenuation”, namely a slowing down or decrease in the rate of reproduction of the pathogen. Such attenuation enables the host's natural defences, such as the host's immune system, to more quickly and effectively eliminate or control the pathogen. Moreover, attenuation of pathogens may result in less prolonged activation of the host's defences which may improve the overall health and physiological performance of the animal.

Patents and patent applications related to the '390 publication have been granted and/or published as follows: AU20072572862; CA265408C; CN101500584B; EP2035018B1; JP2009538837A; JP2013231072A; KR10146209B1; MX2008015200A; and NZ597657. All of the above patents and publications are hereby incorporated by reference in their entirety.

The '390 publication and related patents and/or publications identify elemental sulphur as the active therapeutic ingredient. Sodium lignin sulphonate is described as acting as a catalyst for removing the sulphur atoms from a ring structure thereby making the sulphur atoms available for systemic oxidization and a corresponding reduction in the production of systemic hydrogen protons. However, an inventor of the present disclosure has determined that compositions comprising lignosulfonate are biologically effective for use in pathogenic attenuation even in the absence of elemental sulphur. The present disclosure is therefore directed in some embodiments to compositions which comprise lignosulfonate and are substantially free of elemental sulphur.

The foregoing examples of the related art and limitations related thereto are intended to be illustrative and not exclusive. Other limitations of the related art will become apparent to those of skill in the art upon a reading of the specification and a study of the drawing(s).

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In one aspect, this application relates to the use of a composition comprising lignosulfonate for the prevention and treatment of pathogenic and medical disorders in humans and animals, wherein said composition is substantially free of elemental sulphur. In one particular aspect the composition comprises radically polymerized sodium lignosulfonate.

In another aspect, this application relates to a method of preventing or treating a pathogenic or medical disorder in a human or animal subject comprising administering to the subject an effective amount of a composition comprising lignosulfonate, wherein the composition is substantially free of elemental sulphur.

In another aspect, this application relates to a pharmaceutical composition comprising lignosulfonate, wherein the composition is substantially free of elemental sulphur and is formulated in a dosage for use in the prevention or treatment of a pathogenic or medical disorder in a human or animal subject.

In one aspect this application relates to the use of a composition comprising lignosulfonate for the prevention and treatment of Type I diabetes in humans and animals. In some embodiments the composition alters the balance of serum concentration of Th1/Th2 cytokines as compared to untreated subjects. In some embodiments the composition attenuates the concentration of anti-insulin antibodies in the serum of subjects. In some embodiments the composition increases the serum concentration of one or more cytokines selected from the group consisting of TNF-α IFN-γ, IL-6, and IL-10 in subjects. In some embodiments the composition increases the pancreatic infiltration of CD8+ T cells in subjects.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawing(s). It is intended that the embodiments and figure(s) disclosed herein are to be considered illustrative rather than restrictive.

FIG. 1 is a graph showing proportions of NOD mice that became diabetic when administered with compositions comprising lignosulfonate and variable amounts of elemental sulphur (or no elemental sulphur).

FIGS. 2A-2C are graphs showing the effect of various concentrations of TemStik (identified as Composition 4) on the viability of a mouse beta cell line. Cell viability of mouse beta cells was determined by Trypan blue exclusion dye. Mouse beta cells were seeded at 3×10⁶ cells per well and treated with different concentrations of Composition 4 and incubated for 24 hours under standard culture conditions. FIG. 2A shows results from a first experiment. FIG. 2B shows results from a second experiment. FIG. 2C shows pooled results from the first and second experiments.

FIGS. 3A-3C are graphs showing ATP concentration in mouse beta cells as measured by colorimetric assay. Mouse beta cells were seeded at 3×10⁶ cells per well and treated with different concentrations of Composition 4 and incubated for 24 hours under standard culture conditions. FIG. 3A shows results from a first experiment. FIG. 3B shows results from a second experiment. FIG. 3C shows pooled results from the first and second experiments. ATP levels were measured using an ATP assay kit.

FIGS. 4A-4C are graphs showing proportions of naïve female NOD mice and those that were treated with saline or with a lignosulfonate test compound (LS Compound) that did not become diabetic within 40 weeks of age. FIG. 4A shows weekly blood glucose levels of NOD mice starting at 5 weeks of age until diabetes onset or at 40 weeks of age. FIG. 4B shows average blood glucose levels of female NOD mice upon diabetes onset indicating significant (p<0.05) difference was found between diabetic animals treated with LS Compound and a saline treated group. FIG. 4C shows comparison of *p<0.05 LS Compound treated mice to saline treated mice; and comparison of 5 p<0.05 LS Compound treated mice to naïve mice.

FIGS. 5A-5C shows immunohistochemical analysis of islets in the pancreas of NOD mice. Representative pancreas sections were prepared from LS Compound treated NOD mice that became diabetic and those that remained normoglycemic and stained for insulin and glucagon. All islets in each pancreas tissue sections were analyzed. FIG. 5A shows grading of immune cell infiltrates in pancreatic tissues of diabetic and non-diabetic female NOD mice. FIG. 5B is a graph showing CD8b immunohistochemical analysis of islets in the pancreas. A representative image is shown from 9-15 diabetic and 1-6 non-diabetic mice per group. Endothelial cells were positive for CD8b Scale bar represents 100 μm respectively. FIG. 5C shows comparison of *p<0.05 LS Compound treated group to saline group; and comparison of 5 p<0.05 LS Compound treated group to naïve group. DM; Diabetic mice, NDM; Non-Diabetic Mice.

FIGS. 6A-6B are graphs showing quantification of IL-17, IFN-γ, TNF-α, IL-1β, IL-2, IL-4, IL-6 and IL-10 cytokines in serum of NOD mice at the time of diabetes onset (DM) or at the end of the study (NDM) (40 weeks of age) by ELISA. Data were plotted as mean±SD. Experiments were performed in duplicate per condition. *p<0.05, naïve group compared to saline control group; ^{δ}p<0.05 LS Compound group compared to naïve control group. FIG. 6A shows comparison of cytokines between Diabetic Mice (DM) and Non-Diabetic mice (NDM) from lignosulfonate (LS Compound) treated group. FIG. 6B shows #p<0.05, compared to DM group.

FIG. 7 shows APJ, Apelin and Vanin-1 immunohistochemical analysis of islets in the pancreas of NOD mice. Pancreas sections were prepared from diabetic (DM) and non-diabetic (NDM) female NOD mice treated or not treated with LS Compound for each group and stained for APJ, apelin and Vanin-1. All islets in each pancreas tissue sections were analyzed. A representative image is shown from 9-15 DM and 1-6 NDM mice per group. Scale bar represents 100 μm respectively.

FIG. 8 is graph showing ATP concentrations in mouse beta cells measured by colorimetric assay. Mouse beta cells were seeded at 3×10⁶ cells per well and treated with different concentrations of LS Compound and incubated for 24 hours under standard culture conditions. *p<0.05, compared to untreated group; **p<0.01, compared to untreated group; ***p<0.001, compared to untreated group.

FIG. 9 is a graph comprising a scatter plot showing individual anti-insulin antibody concentrations (in U/mL) for diabetic (DM) and non-diabetic mice (NDM). Anti-insulin antibodies were measured using ELISA assay. The gray line at 1.56 U/mL represents the upper threshold for negative results. The frequency of diabetic mice with anti-insulin antibody above threshold was significantly different from that of the saline control group. *p<0.01 LS Compound treated mice compared to saline treated mice.

FIG. 10 is a graph showing changes in the body weight of NOD mice during the 40-week treatment with LS Compound.

FIG. 11 is an illustration of a typical microtiter plate arrangement for a Trypan Blue Cellular Debris (TBCD) Assay to assess anti-viral activity against HSV-1.

FIG. 12 is an illustration of a typical microtiter plate arrangement for a plaque reduction assay (PRA) to assess anti-viral activity against HSV-1.

FIG. 13 are a series of images showing the effect of lignosulfonate preparations on Verio cells allowed to incubate for 48 hours.

FIG. 14 is a graph showing toxicity of lignosulfonate preparations on Vero cells (TBCD assay results).

FIG. 15 is a graph showing infection inhibition of lignosulfonate preparations on Vero cells (PRA results).

FIG. 16 is a graph showing ISAV titers by log TCID₅₀ of each ISAV treatment group.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Along with cellulose, lignin is one of the primary constituents providing the structure of wood. Lignins are present in significant amounts in plants, accounting for 15-25% (w/w) of herbaceous biomass. They represent a large amount of the waste generated by different industries that use plant matter. Currently, most commercial lignin is obtained as a by-product from lignocellulose treatments performed during pulp and paper processing, in the form of lignosulfonates and kraft lignins. Lignosulfonates are amorphous branched polymers that may contain sulfonated covalently linked phenyl propane monomers and other heterogeneous compounds. In particular, lignosulfonates may contain numerous functional groups, such as carboxylic acid, phenolic hydroxyl, cathechol, methoxyl, sulphonic acid and various combinations of these¹. The precise chemical structure of lignin is not known, both because of its complex polymeric nature and also due to the degree of random coupling involved in the arrangement of the macromolecule. The composition, molecular weight, and amount of lignin differ from plant to plant, with lignin abundance generally decreasing in the order: softwoods>hardwoods>grasses. The components are joined by 13-O-4, 5-5, 13-5, 4-O-5, 13-1, dibenzodioxocin, and 13-13 linkages, among others, of which the 13-O-4 linkage is dominant, accounting for more than half of the linkage structures of lignin².

Some studies show that lignins are involved in different biological activities. These include reducing serum cholesterol by binding to bile acids in the intestine³, and preventing tumor development as demonstrated in rats exposed to an intestinal carcinogen 3,2-dimethyl-4-ami-nobiphenyl and fed a lignin diet^{4 5} It has been shown that the administration of glucose and lignosulfonate induced a delay in glucose uptake and a reduction in blood glucose concentration compared to administration of glucose alone⁶. Further, it has been demonstrated in vitro using Caco-2 cells, where lignosulfonate significantly inhibited the uptake of 2-deoxyglucose. Other potentially beneficial effects of lignosulfonate have been demonstrated, for example that feeding lignosulfonate to KK-Ay diabetic mice suppressed the increase of serum glucose levels compared to the untreated control animals⁷.

Lignosulfonates are commonly used for many different applications, including as binders, pelletizing agents, briquetting agents, anti-caking agents, surfactants, dust suppressants and coagulants. Lignosulfonates are also used as macronutrients in feedingstuffs for animals, including cattle, pigs and chickens. Toxicological studies on lignosulfonates indicate that they are non-toxic. In the literature, the LD50 for lignosulfonates has been reported to be 20,000 mg/kg body weight be ingestion. Materials with LD50 values of 5,000 mg/kg of body weight or greater are considered to be non-toxic (e.g. Material Safety Data Sheet, Ammonium lignosulfonate liquid, Tembec—Chemical Group, accessible online at www.maritimehydroseed.com/images/TDS_MSDS.pdf). Further, lignosulfonates have been approved by the USDA for inclusion in animal feeds.

An inventor of the present disclosure has determined that compositions comprising lignosulfonates when administered in a biological effective dose are useful in the prevention and/or treatment of pathogenic or medical disorders in humans and animals. In some embodiments the compositions may be substantially free of elemental sulphur. In some embodiments the lignosulfonates may be radically polymerized. For example, the lignosulfonates may be radically polymerized by subjecting the lignosulfonates to very high heat.

As described herein, the biological effect of compositions comprising lignosulfonate occurs even in the absence of elemental sulphur in the administered compositions. Without being bound by any particular theory, the inventor(s) believes that the compositions are acting by attenuation of pathogens or other biological agents, enabling the host animal to mount an effective immune or other biological response. For example, the compositions may prevent the pathogen population from growing exponentially in the host animal, thereby enabling the animal to mount an effective immune or other biological response.

As used in this patent application “pathogenic and medical disorders” refers to disorders caused by pathogens or other causes. Pathogenic disorders include disorders caused by pathogens such as bacteria, viruses, prions, protists, fungi and parasitic worms. Medical disorders include disorders resulting in abnormal biological functioning caused by non-pathogens, or only partially caused by pathogens, such as cancer, diabetes mellitus, and reproductive maladies. One particular type of medical disorder is dementia, including Alzheimer's disease. By way of further example, pathogenic and medical disorders may include disorders caused by microbial pathogens; disorders caused by viral pathogens; disorders caused by prions; disorders caused by protists; disorders caused by fungi; disorders caused by parasites; lung and airway disorders; bone, joint and muscle disorders; digestive disorders; hormonal disorders; cancer; auto immune disorders; neurodegenerative disorders; skin disorders; and sexual and reproductive disorders.

One particular complex metabolic disorder is diabetes mellitus. Diabetes mellitus (DM) results from defects in insulin secretion, action, or a combination of both⁸. It is the most common endocrine disorder that affects more than 285 million people worldwide. There are two major classifications of DM. Type 1 DM (T1D) is associated with complete or near total insulin deficiency caused by the autoimmune mediated destruction of pancreatic β-cells, and Type 2 DM (T2D) is associated with variable degrees of insulin resistance, impaired insulin secretion, moderate to severe β-cell destruction and increased hepatic glucose production⁹. Without effective prevention and management programs, further significant rises in diabetes will have consequences on the health and lifespan of the world population (International Diabetes Federation 2019).

There is a growing interest in traditional medicinal plants which produce a variety of compounds that have therapeutic properties^{10 11 12}. Besides drugs, several species of plants have been reported to have anti-diabetic activity^{10 11 12}. Many drugs commonly used today to control diabetes are structurally derived from natural compounds that are found in traditional medicinal plants. For example, the development of the drug metformin can be traced to the traditional use of Galega officinalis to treat diabetes¹¹. Many plant products are rich in polyphenolics including tannins and flavonoids, which are group of compounds with diverse chemical structure, characteristics, and are widely recognised as naturally occurring antioxidants¹². Several reports revealed that compounds in their natural formulations are more active than isolated form, since they contain both dotes and antidotes¹³. Lignin is a very promising candidate for the treatment of diabetes and other disorders due to the presence of phenolic and aliphatic hydroxyl groups in its structure as discussed above¹⁴. As described herein, the anti-diabetic potential of lignosulfonates has been demonstrated using a mouse model of T1D.

Compositions comprising lignosulfonates are generally available from a wide variety of sources, including businesses involved in pulp and paper processing. By way of an exemplary example, a composition comprising radically polymerized sodium or ammonium lignosulfonates is or has been available from the Tembec—Chemical Group, acquired by Rayonier Advanced Materials, and has been sold under the trademark ARBO®TemStik (hereinafter “TemStik”). A chemical analysis of TemStik in powder form has confirmed that it does not include a measurable amount of elemental sulphur. In particular, the TemStik composition contained less than the reportable detection limit of sulphur (elemental) in a chemoanalytic analysis where the detection limit was 100 mg/kg.

The TemStik composition comprising radically polymerized lignosulfonates may also comprise measurable amounts of tannic acid and aldehydes such as formaldehyde, acetaldehyde and propionaldehyde. Without being bound by any particular theory, the inventors believes that one or more of the above constituents could provide a biological effect in vivo, either directly or acting synergistically with other multi-molecular constituents. Further, without being bound by any particular theory, the inventors believes that the TemStik composition comprising radically polymerized lignosulfonates may function as an ATP inhibitor to effect broad-spectrum pathogenic attenuation at biologically effective dosages.

### Example 1—Administration of TemStik to Livestock

A supply of TemStik in powder format was purchased in 25 kg bags. Two bags of TemStik, namely 50 kg, was mixed with 4 metric tons, namely 4,000 kg, of animal feed. This ratio corresponds to 12.5 g of TemStik per kg of feed. The animal feed comprised 2000 kg of barley, 1000 kg of wheat and 1000 kg of peas. The animal feed supplemented with TemStik was fed to livestock, principally swine. In this example 100 swine each weighing on average 50 kg were fed 4 metric tons of animal feed over 4 days in a livestock pen. Thus each swine consumed on average 10 kg of animal feed comprising 125 g of TemStik per day. That is, for every 50 kg of animal weight, approximately 125 g of TemStik was administered per day. This corresponds to a daily dosage of 2.5 g of TemStik per kg of livestock weight. Based on experience administering feed supplements to livestock, the inventor(s) believes that minimum and maximum daily dosage may vary significantly from the dosage provided in this example while maintaining biological efficacy. For example, in some embodiments the minimum and maximum daily dosage may vary by 20% from the dosage provided in this example. Thus in such embodiments the daily dosage of TemStik per kg of livestock weight could vary within the range of approximately 2-3 g of TemStik per kg of livestock weight. In another embodiment the daily dosage of TemStik per kg of livestock weight could be reduced by approximately 50% from the dosage provided in this example while maintaining biological efficacy.

The livestock was monitored over an extended period of time and exhibited excellent health without the need to administer prophylactic or therapeutic antibiotics or other veterinary drugs. In particular, the livestock exhibited excellent viability, resistance to pathogenic and medical disorders, and excellent reproductive performance. For example, although the livestock were not administered with any vaccines, anti-microbial agents or other anti-parasitic agents, and were subject to periodic inspection by Canadian federal government regulators, very few animals or animal organs were condemned due to parasitic or other animal diseases. The number of swine slaughtered and subject to inspection per year was approximately 5,000.

### Example 2—Use of TemStik in Mice to Inhibit the Onset of Type 1 Diabetes

The biological efficacy of TemStik was investigated in an animal model of Type 1 diabetes using non-obese diabetic (NOD) mice. In this example TemStik was compared to other compositions comprising both lignosulfonates and elemental sulphur in varying proportions as well as to a saline control. The mice were five-week old female NOD mice. One intraperitoneal injection of TemStik at 5 mg/ml was administered to NOD mice on the first day of the experiment followed by a second injection one day later. On the second day of the experiment TemStik was provided to the mice in their drinking water at a concentration of 1 mg/ml. The other test compositions (Compositions 1-3) and saline control were administered in a similar manner, except that Composition 3 was provided to mice in their drinking water at concentrations of 0.04 mg/ml (Low) or 0.2 mg/ml (High).

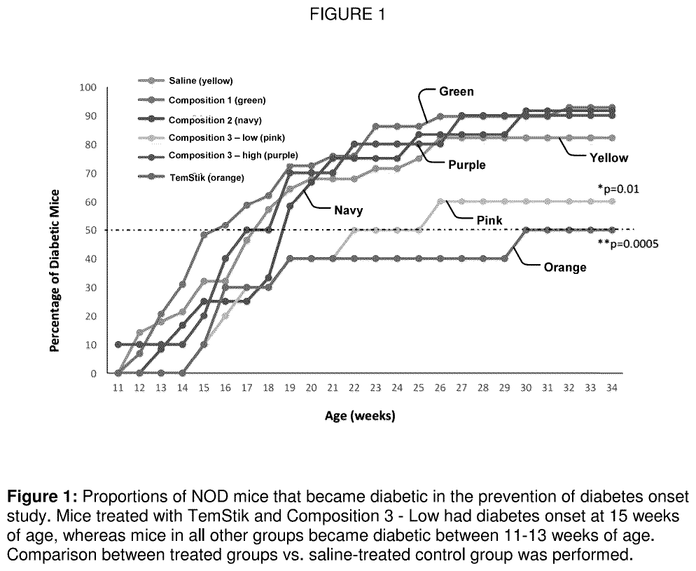

The proportion of NOD mice that became diabetic in this example is graphically illustrated in FIG. 1. In the case of the mice administered with TemStik, 5 of the 10 mice treated remained non-diabetic after 30 weeks of treatment. The time of diabetes onset in weeks for individual mice administered with TemStik was as follows: 15, 16(×2), 19, 30, 34 and 35(×4). This corresponds to Mean±SEM (weeks) of 27.00±2.91. This experimental data demonstrates that TemStik was significantly more effective in preventing the onset of diabetes than the other tested compositions and the saline control. The test composition that was the next most effective in preventing the onset of diabetes was the composition containing lignosulfonate but having the smallest concentration of elemental sulphur (Composition 3—Low). This experiment demonstrates that compositions comprising lignosulfonate but substantially free of elemental sulphur are effective in preventing the onset of Type 1 diabetes.

### Example 3—ATP Inhibition

As indicated above, it is believed the TemStik composition comprising radically polymerized lignosulfonates may function as an ATP inhibitor, likely via ATPase enzymes embedded in the mitochondrial membrane, to effect broad-spectrum pathogenic attenuation at biologically effective dosages.

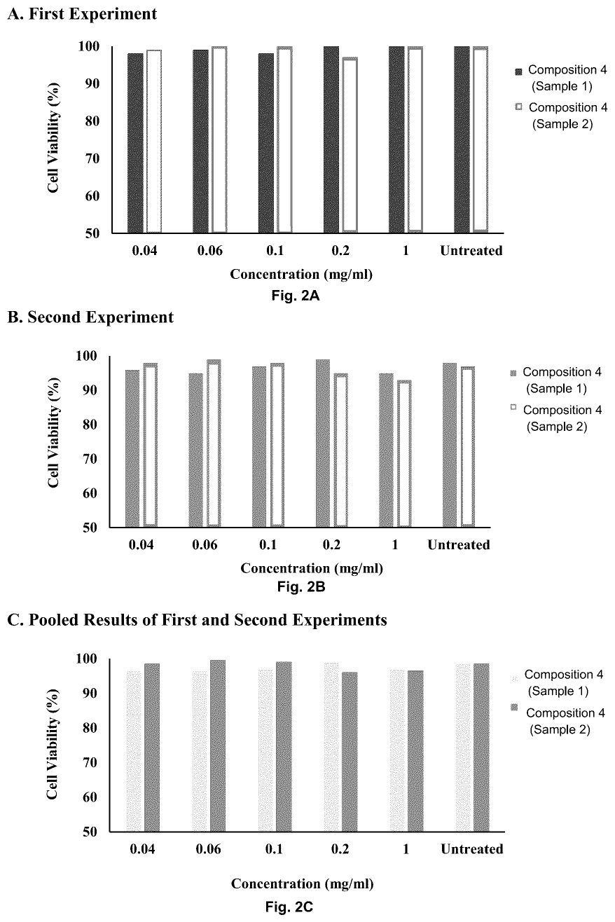

FIGS. 2A-2C show the effect of various concentrations of TemStik (Composition 4) on the viability of a mouse beta cell line. Two different samples of Composition 4 were tested. Regardless of the sample source and dose of Composition 4, the viability of the beta cells treated with Composition 4 remained high and comparable to those observed in untreated cells.

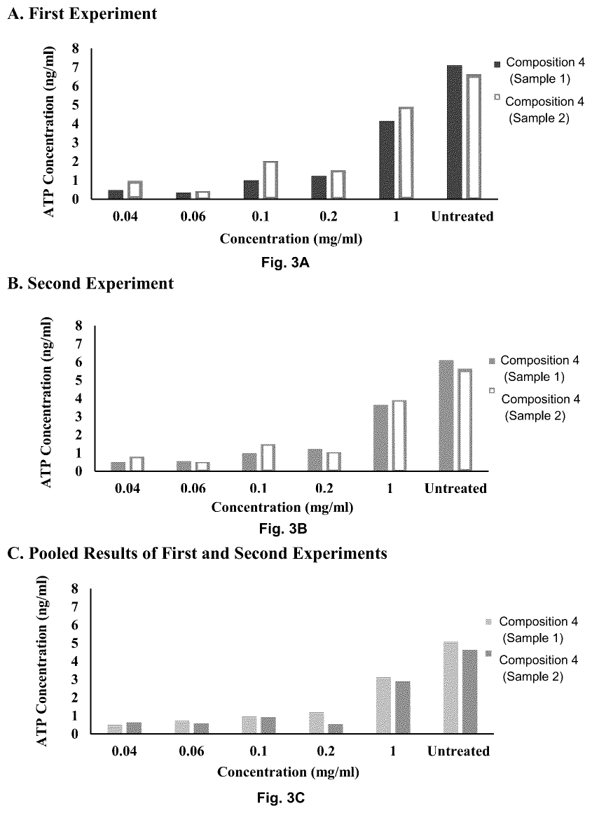

FIGS. 3A-3C show the effect of various concentrations of Composition 4 on ATP concentration in mouse beta cells. The level of ATP concentration in cells treated with Composition 4 are significantly lower compared to those detected in untreated cells. ATP levels were measured using an ATP assay kit.

### Example 4—Swine Serological Study

A serological study of swine fed with animal feed supplemented with TemStik was conducted. The daily dosage of TemStik per kg of livestock weight was approximately 50% of the dosage described in Example 1 above. Blood samples from 30 individual animals was collected and analyzed. The analysis showed that the swine tested positive for antibodies to various pathogenic agents. The detected antibodies included influenza H3N2 antibody; Mycoplasma hyopneumoniae antibody; and influenza H1 antibody. The following antibodies were not detected above the detection limit: PRRSV antibody and TGEV-PRCV antibody. The absence of the undetected antibodies was somewhat surprising since such antibodies were present in earlier tests of the swine herd, e.g. ancestor animals.

The swine cohort in question were not administered with any vaccines, anti-microbial agents, anti-parasitic agents or other veterinary drugs. Despite the presence of antibodies suggesting exposure to various pathogenic agents, the swine cohort exhibited excellent viability, resistance to pathogenic and medical disorders, and excellent reproductive performance. Further, following slaughter of the swine, very few animals or animal organs were condemned.

### Example 5—Wild Boar Study

Wild boar fed with animal feed supplemented with TemStik were slaughtered in a Canadian Food Inspection Agency (CFIA) regulated plant in Manitoba, Canada. The daily dosage of TemStik per kg of livestock weight was approximately 50% of the dosage described in Example 1 above. The wild boar in question were not administered with any vaccines, anti-microbial agents, anti-parasitic agents or other veterinary drugs. In the Manitoba plant a total of 2,783 head of wild boar were slaughtered over a period of several months and only 19 (0.7%) of the slaughtered animals were condemned.

Wild boar fed with animal feed supplemented with TemStik were slaughtered in a Canadian Food Inspection Agency (CFIA) regulated plant in Quebec, Canada. The daily dosage of TemStik per kg of livestock weight was approximately 50% of the dosage described in Example 1 above. The wild boar in question were not administered with any vaccines, anti-microbial agents, anti-parasitic agents or other veterinary drugs. In the Quebec plant a total of 800 head of wild boar were slaughtered over a period of several months and none of the slaughtered animals were condemned.

### Example 6—Anti-Diabetic Effect of Lignosulfonate in NOD Mouse Model of Type 1 Diabetes Mellitus

### Materials and Methods

The biological efficacy of lingosulfonate was further investigated in an animal model of Type 1 diabetes (T1D) using non-obese diabetic (NOD) mice and in mouse cell lines. In this example, the effect of lignosulfonate on blood glucose levels, insulin and immune cell infiltrates, cytokine production, pancreatic tissue immunochemistry, ATP inhibition and anti-insulin antibody concentration was investigated.

### Lignosulfonate Compound

A commercially available ammonium lignosulfonate, hereinafter referred to as the LS Compound, was purchased from Tembec Inc., (Quebec, Canada). The LS Compound has been sold under the trademark ARBO®TemStik. The LS Compound was obtained in powder form and was used as received without further purification.

### Animals

Inbred female non-obese diabetic (NOD/MrkTac) mice were purchased from Taconic Farms (Germantown, NY) at 4 weeks of age and were acclimatized in Health Sciences Laboratory Animal Services facility of the University of Alberta for one week. All mice were housed under pathogen-free conditions, in individually vented caging with 12 hour light/dark cycle and were provided with standard laboratory food and water ad libitum. Animals were cared for according to the guidelines of the Canadian Council on Animal Care and use of animals in this study was approved by the University of Alberta's Animal Care and Use Committee, Health Sciences.

### Treatment

At 5 weeks of age, mice were randomly assigned to one of the three groups: Saline treated control group, LS compound treated group or untreated Naïve mice group. Mice received 2 consecutive days of intraperitoneal (IP) injections of saline or LS Compound (5 mg/ml)) followed by respective oral treatment of 1.0 mg/ml in their drinking water. The water was changed and weighted 3 times/week and was offered ad libitum to the mice. Naïve mice did not receive IP injections. Food, body weights and blood glucose levels were monitored weekly. Non-fasting morning blood glucose concentrations were measured by tail vein prick using a OneTouch Ultra 2 blood glucose meter (Life Scan, Switzerland) weekly until 40 weeks of age or until the mice became diabetic. Diabetes was defined if the mouse exhibited two consecutive days of non-fasting blood glucose levels above 17 mmol/1. That is, if a mouse exhibited two consecutive days of non-fasting blood glucose above 17 mmol/1 it was subsequently euthanized and samples were collected. Mice that did not develop diabetes at 40 weeks of age were euthanized and samples were also collected.

### Immunohistochemical Staining

Pancreatic tissue was removed from mice that became diabetic or at the end of study (40 weeks of age) if they remained non-diabetic. Tissues were placed in z-fix for 24 hrs then washed in 70% ethanol, processed for dehydration and paraffin-embedded. Tissues were cut into five-micron sections and stained for insulin, glucagon, CD4, CD8, CD19, F4/80, apelin, APJ, Vanin-1 and IgG antibody. To detect the presence of insulin, the sections were deparaffinized with histoclear and graded ethanol solutions. Tissue samples were then stained with guinea pig anti-mouse insulin antibody (1:1000 dilution; Dako, Carprinteria, CA) or mouse anti-glucagon antibody (1:5000 dilution; Sigma-Aldrich, Oakville, ON) for 30 minutes followed by the addition of biotinylated goat anti-guinea pig IgG secondary antibody (1:200 dilution; Vector Laboratories, Burlington, CA) for insulin and biotinylated goat anti-mouse IgG secondary antibody (1:200 dilution; Vector) for glucagon. For staining CD4, CD8, CD 19, F4/80, apelin, Vanin-1 and enterovirus-71 antigen heat mediated antigen retrieval immunohistochemistry standard protocol was performed on tissue sections that were deparaffinized. Antigen was retrieved by microwaving the tissue sections for 10 minutes in sodium citrate buffer of pH 6.0. Rat anti-CD4 antibody (1:100 dilution, Thermo Fisher, Ottawa, ON), rabbit anti-CD8b antibody (1:100, Cedarlane, Burlington, ON), rabbit anti-CD19 antibody (1:100, Abcam, Toronto, ON), rat anti-F4/80 antibody (1:100, Abcam) or enterovirus-71 antigen (1:600, Thermo Fisher) was applied to the respective tissue section and left overnight in 4° C. Appropriate IgG secondary antibody was added and incubated for 30 minutes at room temperature. For detection of APJ the sections were stained with rabbit anti-APJ antibody (1:150 dilution; Abcam) using the same protocol except that the tissue samples were incubated for 90 minutes followed by the addition of biotinylated goat anti-rabbit IgG secondary antibody (1:200 dilution, Vector). Rabbit anti-apelin antibody (1:1000, Abcam), and rabbit anti-vanin-1 antibody (1:1000, Abcam) were used to detect apelin and Vanin-1 following the same protocol except that the antigen was retrieved by microwaving for 10 minutes in Tris-EDTA buffer of pH 9.0. Avidin-biotin complex/horseradish peroxidase (ABC/HP; Vector) and 3,3-diaminobenzidinetetrahydrochloride (DAB; BioGenex, San Ramon, CA) were used to produce a brown colour positive reaction. All sections were counterstained with either Harris' hematoxylin and eosin (insulin and glucagon) or just Harris' hematoxylin (CD4, CD8, CD19, F4/80, apelin, APJ, Vanin-1, IgG and enterovirus-71). For the detection of anti-IgG autoantibody, pancreas sections of mice were incubated with their own serum (1:128 dilution) collected when diabetes was confirmed or at the end of the study (40 weeks of age) for 30 minutes, followed by the addition of biotinylated goat anti-rabbit IgG secondary antibody (1:200, Vector).

### Assessment of Pancreatic Islets

Insulitis in the pancreas of NOD mice was scored using insulin/glucagon images, under light microscopy following the grading: 0, no insulitis and no immune cell infiltration; 1, insulitis affecting less than 25% of the islet; 2, insulitis affecting 25-50% of the islet; 3, more than 50-75% islet and 4, >75% islet was infiltrated. Eighty-five to 257 islets were scored for insulitis in each group by individuals blinded to treatment groups.

### Cytokine Measurement

Blood samples were collected from diabetic mice or mice that remained non-diabetic at the end of the study (40 weeks of age). Serum from blood samples were collected by allowing the blood to clot at room temperature for 30 minutes, then centrifuged at 2,800×g for 20 minutes in a refrigerated centrifuge. Serum was then transferred into an Eppendorf tube and stored at −80° C. until use. Cytokine levels were measured in the serum using multiplex assays manufactured by Meso Scale Discovery (MSD; Gaithersburg, MD). Each well of the 96-well plate-based assays contained antibodies to IFN-γ, TNF-α, IL-6, IL-10, IL-1, IL-4 and IL-2. The assay was performed following the manufacturer's instructions. Briefly, plates were washed three times with wash buffer followed by the addition of 50 μl serum sample to each well. To determine the precise concentration, dilutions of the supernatant were tested in duplicates. The plates were incubated at room temperature with shaking for 24 hours. After incubation plates were washed three times with wash buffer and a 25 μl of detection antibody solution was added to each well followed by an incubation of 2 hours at room temperature with shaking. After incubation plates were washed 3 times with wash buffer followed by an addition of 150 μl 2× read buffer to each well. The lower detection limits of the assays were 0.390 pg/ml for IFN-γ, 0.980 pg/ml for TNF-α, 7.61 pg/ml for IL-6, 19.8 pg/ml for IL-10, 0.72 pg/ml for IL-1, 2.58 pg/ml for IL-4 and 1.03 pg/ml for IL-2. Absorbance was measured at 450 nm using an En Vision multilabel reader 2104 (PerkinElmer, ON). Cytokine concentrations in the test samples were calculated based upon standard curves generated with known concentrations of recombinant mouse cytokines.

### ATP Measurement

To measure ATP levels, mouse beta cells (Beta-Tc-tet cell line, ATCC) were seeded at 3×10⁶ cells per well and treated with different concentrations (0.04, 0.06, 0.1, 0.2 and 1 mg/mi) of LS Compound and incubated for 24 hours under standard culture conditions. The in vitro cell line study for measuring ATP levels was separate from the animal study using NOD mice described above After 24 hours of incubation cells were washed 3 times with 1×PBS and pelleted at 800×g, the cell pellets were immediately stored in liquid nitrogen. Total ATP levels were measured using the ATP assay kit (Abcam), and following the manufacturer's protocol. Briefly, cells were thawed on ice and 100 μl of ATP assay buffer was added to each sample and cells were homogenized quickly by pipetting up and down a few times. Samples were then centrifuged for 5 minutes at 4° C. at 13,00×g in a micro centrifuge. Supernatant was neutralized using the deproteinizing kit (Abcam). For each sample 1 replicate tube was prepared containing ATP reaction mix and a sample. The background control mix was added to the control sample wells. The samples were mixed and incubated for 30 minutes at room temperature. Quantification of ATP assay was performed by colorimetric measurements (ODmax=570 nm) using an En Vision multilabel reader 2104 (PerkinElmer, ON). ATP concentrations in the test samples were calculated based upon standard curves generated with known concentrations of pure ATP.

### Quantification of Anti-Insulin Antibodies

Serum samples were analyzed for the presence of anti-insulin antibodies following manufactures instructions (MyBioSource, CA). Briefly, plates were pre-coated with mouse anti-Insulin antigen. The standard was diluted from a 100 U/ml stock solution provided with the kit and serially (log 2) diluted from 100 U/ml to 1.56 U/mL. Test serum samples were diluted at 1:10 dilution. The prepared standard and the test serum samples was added to the respective wells and the plates were incubated for 90 minutes at 37° C. Plates were washed with wash buffer twice using 250 μl of buffer, with shaking between each step and an antigen was applied at a concentration of 1:100 (100 μl/well) and incubated for 60 minutes at 37° C. Plates were washed three times, and 100 μl/well of enzyme conjugate was added and incubated for 30 minutes at 37° C. Plates were washed with wash buffer for five times and a 100 μl/well of colour reagent was applied and incubated for 30 minutes at 37° C. and a colour reagent C was added and the plates were allowed to develop the colour reaction. The OD of each well was determined by measuring the absorbance at a wavelength of 450 nm using a Bio-Tek EL-3 11 microplate autoreader (Bio-Tek Instruments, Inc., Winooski, VT). Antibody concentrations were determined by multiplying the dilution factor of the sample to correct for the final concentration.

### Results

Effect of Lignosulfonate on Blood Glucose in NOD Mice

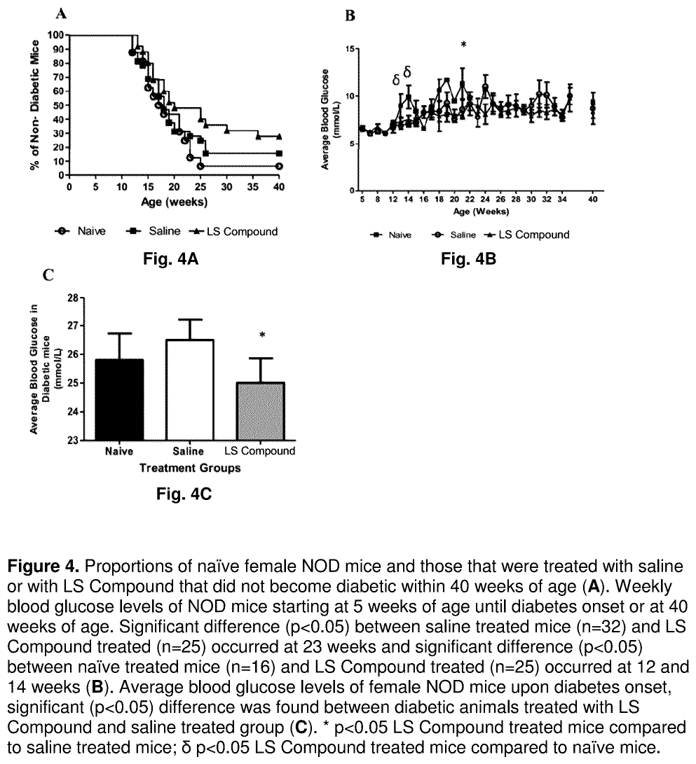

In this example the anti-diabetic effects of lignosulfonate (LS Compound) was investigated in vivo using NOD mice, a model for Type 1 diabetes. Blood glucose levels and body weights of NOD mice were measured once a week until the end of the study period. The mice treated with the LS Compound delayed the onset of diabetes and the median survival times for the naïve, saline treated control and LS Compound treated mice were 16.5, 18.5 and 20 weeks, respectively (FIG. 4A). Significant difference (p<0.05) between saline treated mice (n=32) and LS Compound treated (n=25) occurred at 23 weeks and significant difference (p<0.05) between naïve treated mice (n=16) and LS Compound treated (n=25) occurred at 12 and 14 weeks. The median survival rate of the mice treated with LS Compound was 3.5 weeks longer than the naïve group. This difference is statistically significant (p=0.043). The percentage of mice that became diabetic at 40 weeks of age was significantly (p<0.0001) lower in LS Compound treated mice (68%) compared to the naïve group (94%) and the saline treated group (84%) (Table 1, below). Consistent with the reduced disease incidence, the LS compound treated mice exhibited significantly lower mortality rate than the saline (p<0.05) and the naïve (p<0.01) treated group at 40 weeks of age (Table 2, below). In naïve treated group, the average blood glucose level rapidly increased after 12 and 20 weeks of age and in the saline treated group the average blood glucose increased at 24 weeks of age, whereas in LS Compound treated group this increase was suppressed (FIG. 4B). The mean blood glucose levels of mice treated with the compound that became diabetic (25.0 mmol/L±0.85, n=32) was significantly lower (p<0.05) compared to the saline treated control group (26.5 mmol/L±0.70, n=25). However, there was no significant changes found in the mean blood glucose levels between naïve control group and the LS Compound treated group. During the 40 weeks of study there was no significant difference found in the food intake between the control groups and the LS Compound treated groups. Based on these observations, it was concluded that LS Compound treated NOD mice were rendered diabetes resistant to a significant extent, whereas the saline treated and the naïve mice did not affect the development of T1D.

TABLE 1
Proportions of diabetic NOD mice treated with lignosulfonate
(LS Compound) compared to saline-treated & naive mice

| | | | | | Age | | |
|---|---|---|---|---|---|---|---|
| | | % of | | | (weeks) at | | |
| | | Diabetic | | | which | | |
| | | Mice | | | 50% | | p |
| | | (at 40 | p value | p value | of mice | p value | value |
| | | weeks of | vs | vs | became | vs | vs |
| Group | n | age) | Saline | Naive | diabetic | Saline | Naive |
| Naive | 16 | 94 | p = | N/A | 17 | p = | N/A |
| | | | 0.10 | | (50%) | 0.75 | |
| Saline | 32 | 84 | N/A | p = | 18 | N/A | p = |
| | | | | 0.10 | (53%) | | 0.75 |
| LS | 25 | 68 | p = | p < | 20 | p = | p = |
| Compound | | | 0.03 | 0.0001 | (52%) | 0.53 | 0.34 |

TABLE 2
Incidence of diabetes in female NOD mice treated
with lignosulfonate (LS Compound)

| | | Time of | | | |
|---|---|---|---|---|---|
| | | Diabetes | | p value | p value |
| | | Onset | Survival | vs | vs |
| Group | n | (weeks) | (%) | Saline | Naive |
| naive | 16 | 12(×2), 14, 15(×3), 16, | 6 | p = 0.89 | N/A |
| | | 17, 18, 21(×2), 22, | | | |
| | | 23(×2), 25 | | | |
| Saline | 32 | 12(×4), 13, 14(×2), | 16 | N/A | p = 0.89 |
| | | 15(×3),17(×4), 18(×3), | | | |
| | | 19(×3), 20(×2), 23, | | | |
| | | 25, 26(×3) | | | |
| LS | 25 | 13(×2), 14, 15(×2), | 28 | P < 0.05 | P < 0.01 |
| Compound | | 16(×3), 18(×2) | | | |
| | | 19(×2), 20, 25(×2), | | | |
| | | 26, 30 36 | | | |

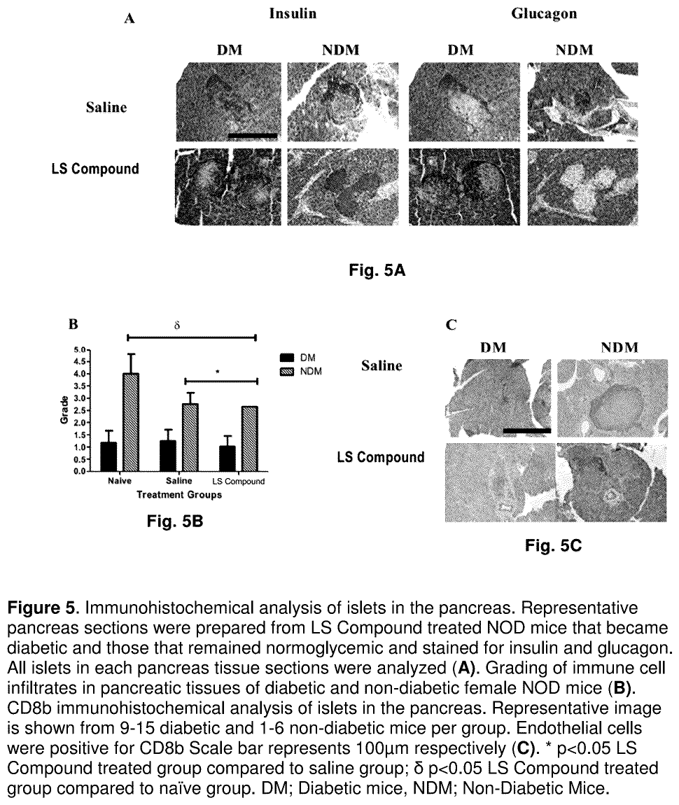

To assess the severity of insulitis in mice treated with the LS Compound, the immune cell infiltrates in the islets of diabetic or non-diabetic mice were determined. H&E staining of pancreatic sections revealed that the insulitis incidents for the mice treated with saline were significantly (p<0.05) lower compared to the LS Compound treated group (FIG. 5A). Saline treated mice only showed ˜10% of islets with peri-insulitis, while ˜90% of islets from LS Compound treated mice presented infiltration. This was also the case for the insulitis grade, the severity of insulitis being graded as one of four levels in each islet, and more glucagon positive islets were observed in all the treatment groups in both diabetic and non-diabetic group (Table 3, below). Overall, a trend of lower cell infiltrate was observed in diabetic mice compared to non-diabetic mice. The cell infiltrate in the LS Compound treated mice that remained normoglycemic was significantly lower (p<0.05) compared to the saline treated and the naïve control non-diabetic mice (FIG. 5B).

Detailed immunohistochemical analyses of the population of infiltrated immune cells in pancreatic islet samples revealed less severe infiltration of CD4, F4/80 and CD19 positive cells in LS Compound treated mice. Comparison of these profiles in mice that became diabetic and mice that remained normoglycemic did not reveal any dramatic difference between the LS Compound treated and the saline treated mice. However, a notable increase in the infiltration of CD8+ T cell numbers was observed in the LS Compound treated non-diabetic mice at 40 weeks of age indicating that these cells may have a relevant role in the onset of Type 1 diabetes (FIG. 5C). The infiltrations of cells were found in both the exocrine and endocrine tissue, cell densities were calculated in both the compartments. Furthermore, analysis of CD8+ T cells individual density values showed significant differences in CD8+ T cell infiltration between saline treated and the LS Compound treated non-diabetic mice (FIG. 5C). Since the interaction between CD4+ and F4/80 macrophages is essential for the initiation of immune responses, correlation between these cell types was analyzed. However, no difference in the expression patterns of these molecules between the study groups was detected.

TABLE 3
Quantification of insulin, glucagon, and cell infiltrate
in islets of diabetic female NOD mice,
treated or not treated with LS Compound.

| | | | | | Islets | |
|---|---|---|---|---|---|---|
| | | | | | Positive | |
| | | Total | | | for | |
| | Number | Number | Insulin | Glucagon | both | |
| Treatment | of | of | Positive | Positive | Insulin & | Infiltrate |
| Groups | Mice | Islets | Islets | Islets | Glucagon | Grading |
| Saline | 5 | 41 | 5 | 41 | 5 | 0.7 |
| Naive | 1 | 1 | 0 | 1 | 0 | 3.0 |
| LS | 6 | 88 | 55 | 75 | 47 | 1.1 |
| Compound | | | | | | |

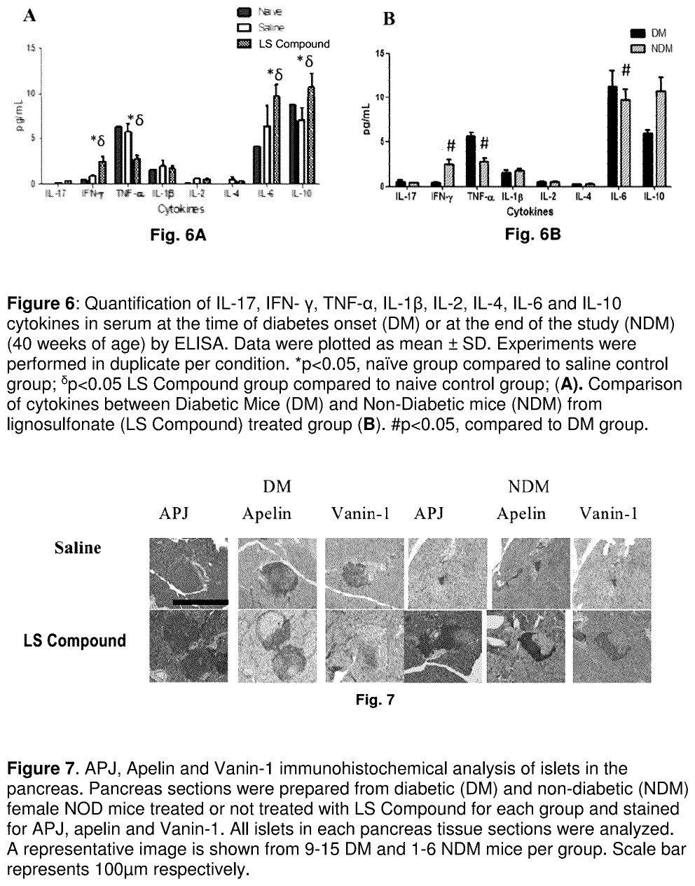

To investigate the immunomodulatory effects of lignosulfonate (LS Compound) on T1D, IFN-γ, IL-17, TNF-α, IL-1β, IL-2, IL-4, IL-6 and IL-10 cytokines were quantified in serum collected at the time of diabetes onset (DM) or at the end of the study (40 weeks of age; NDM). Mice treated with LS Compound showed a significant increase of INF-γ, IL-6 and IL-10 and a significant decrease in TNF-α (p<0.05) compared to the saline treated and the naïve untreated mice (FIG. 6A). INF-γ, IL-6, TNF-α, and IL-10 cytokines differ significantly between compound treated mice that became diabetic and those that remained normoglycemic (FIG. 6B). This observation is reflected in the increased number of CD8+T cells in the immune cell population infiltrating the islets of non-diabetic mice. IL-17, IL-1βIL-2, and IL-4 cytokines were detectable in the serum. Thus, LS Compound treated mice had increased insulitis due to the increased number of infiltrated cells than the saline treated mice, and the Th1/Th2 balance in the infiltrated leukocytes was markedly different between the diabetic and non-diabetic mice treated with compound indicating that the balance between Th1/Th2 cytokines may correlate with the protection from T1D.

### Immunohistochemistry of Apelin, Apelin Receptor, Vanin-1 and Auto IgG Antibody

Expression of apelin and its receptor APJ, and Vanin-1 were examined in the pancreatic tissues by immunohistochemistry. Immunohistochemistry of pancreas tissue sections revealed the positive staining for apelin, APJ and Vanin-1 in saline and compound treated mice that remained normoglycemic. While a strong expression of APJ was observed in diabetic saline treated mice. In the LS Compound treated diabetic and non-diabetic mice, pancreas tissue sections demonstrated a strong expression of APJ. In addition, the expression of Vanin-1 was stronger in LS Compound treated mice that did not become diabetic (FIG. 7). The presence of auto IgG antibody on pancreas tissue sections was also determined and quantified.

### Effect of Lignosulfonate on ATP

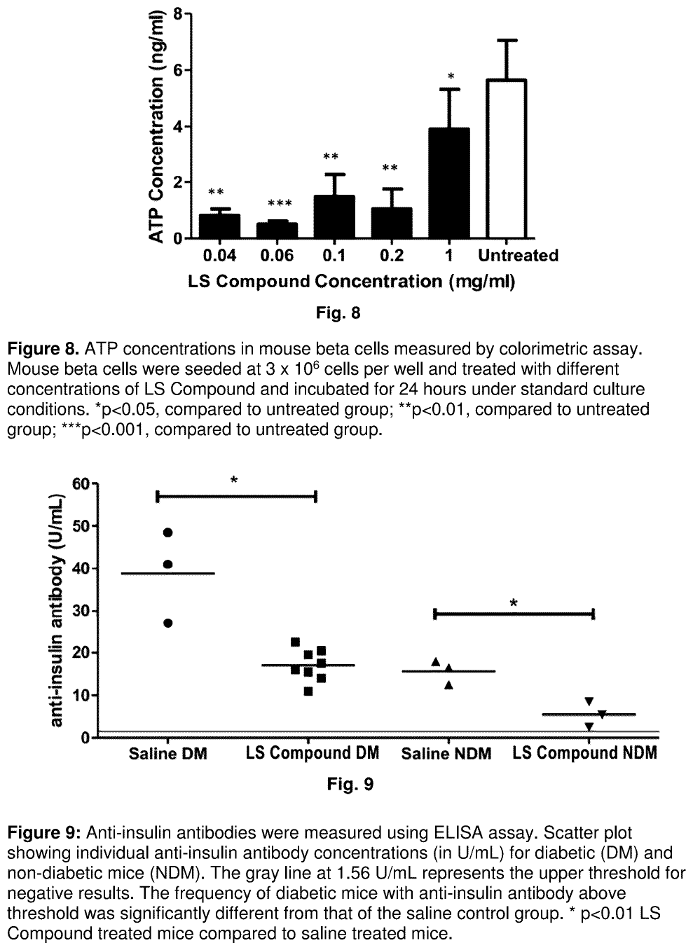

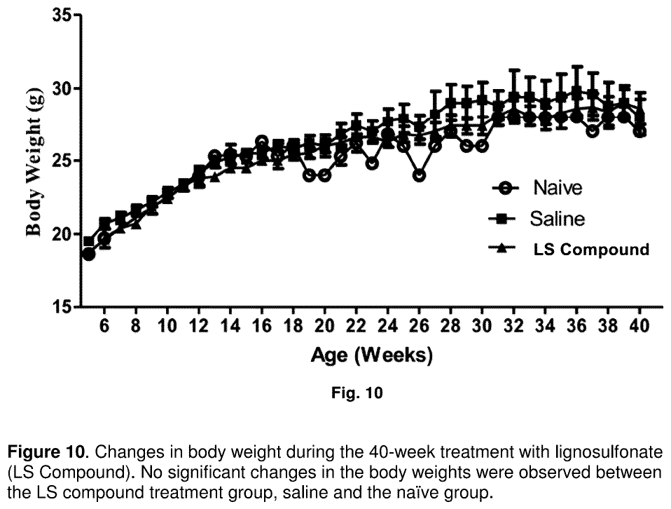

The role of lignosulfonate in insulin secretion on ATP inhibition was investigated using Beta TC-tet cells, a mouse pancreatic E-cell line, as a model. As shown in FIG. 8, ATP inhibition of lignosulfonate was significantly reduced at concentrations of 0.04, 0.06, 0.1, 0.2 and 1 mg/ml during 24 hours of incubation. However higher inhibition was observed at 0.06 mg/ml. The inhibitory effect of ATP was not observed in the absence of LS Compound suggesting that the effect was dependent on the concentration of LS Compound, i.e. ATP inhibition is dose dependent.

### Effect of Lignosulfonate on Anti-Insulin Antibody

The concentration of anti-insulin antibody was significantly lower (p<0.01) in the LS Compound treated mice for both the DM and the NDM compared to the saline control treated group (FIG. 6). Indicating that LS may be useful in reducing, but not suppressing the anti-insulin antibodies, thus improving their hypoglycemia control, and enabling the use of lower doses as a consequence.

### Summary of Findings

The beneficial effects of lignosulfonate (LS Compound) for use in the treatment of T1D was demonstrated in this example. In particular, this example demonstrates that (i) lignosulfonate treatment delayed the onset of Type 1 diabetes in NOD diabetic mice as indicated by the lower blood glucose levels in LS Compound treated mice compared to the saline treated and the naïve mice. (ii) LS Compound treatment suppressed the increase in the number of cell infiltrate in the LS Compound treated mice that remained normalglycemic compared to the saline treated and the naïve normoglycemic mice and (iii) LS Compound treatment increased the expression INF-γ, and IL-10 and a decreased TNF-α cytokines, which is reflected in the increased number of CD8+ T cells infiltration in the islets of non-diabetic mice.

Studies have shown that in children with T1D, a decreased secretion of cytokines (IL-6, IL-10, IL-13, IL-17, IFN-γ, and TNF-α) were reported in comparison to healthy children and a decreased Th1 immune responses at the onset of T1D^{15 16 17 18}. The low cytokine secretions could be an effect of exhaustion of the immune system following a strong immune activation prior to the onset of T1D and therefore most of the f3 cells are destroyed and generate less cytokines^{19 20}. In this example a delay in the onset of T1D and an increased IL-10 response in non-diabetic mice was observed. This observation is confirmed by a previous study that in healthy individuals a Tr1-like (IL-10) immune responses favor survival of f3 cells and thus delays the onset of disease^{19 20}.

It has for long been debated that enteroviral infections increase in frequency in newly diagnosed T1D patients. Enteroviral infections have been reported to be frequent in children and significantly elevated in children diagnosed with T1D than in nondiabetic children²¹. In this example, increased detection of enteroviral infection was observed in the mice that were not treated with the LS Compound. It has recently been shown that monocytes represent an additional cellular target for C-peptide anti-inflammatory activity²². Monocytes are pivotal cells in inflammatory responses as they serve as the principal reservoir of proinflammatory cytokines, e.g., TNF-α. These cells are the first to be engaged in nonspecific immune responses such as those triggered by environmental factors, e.g., by infections. Thus, monocytes can be one of the sources of the release of the proinflammatory cytokine TNF-α in mice with an infection earlier the onset of T1D.

Circulating autoantibodies are a hallmark of autoimmune disease, including T1D^{23 24 25 26}; however, there is a debate regarding the frequency of autoantibodies and their usefulness as a maker for T1D²⁷. Antibodies to insulin (IAA), glutamic acid decarboxylase (GAA or GAD) and protein tyrosine phosphatase (IA2 or ICA512) have all been defined and can be present years before the development of clinically significant hyperglycemia, and the presence of persistently positive and multiple antibodies is highly predictive for the development of T1D^{28 29}.

ATP overproduction is a potential risk factor for the insulin hypersecretion in β-cells. In physiologic conditions, insulin is secreted by β-cells upon glucose challenge through an elevation in ATP production by mitochondria from glucose. ATP may use multiple signaling pathways in the regulation of insulin action. The mechanism by which Metformin is believed to improve insulin sensitivity by the activation of the AMPK pathway and act directly to reduce insulin by inhibiting the ATP production^{30 31 32 33}, Given that hyperinsulinemia is a risk factor for insulin resistance^{34 35 36}, the effect of the LS Compound on ATP production was studied in cell culture model and it was observed that the LS Compound inhibits ATP production. The results of this example suggest that effect of ATP inhibition by the LS Compound is dose dependent and the lower the dose of the LS Compound the higher the ATP inhibition.

The experiments of this example demonstrated that lignosulfonate treated NOD mice were significantly resistant to the onset of diabetes as compared to saline treated and naïve untreated mice. Further, lignosulfonate treated mice showed a significant increase in serum concentration of some cytokines, namely IFN-γ, IL-6, and IL-10, and a significant decrease in TNF-α compared to the saline treated and naïve untreated mice. Moreover, the level of cytokines TNF-α IFN-γ, IL-6, and IL-10 differed significantly between LS Compound treated mice that become diabetic and those that remain normoglycemic. This observation is reflected in the increased number or CD8+ T cells in the immune cell population infiltrating the islets of the non-diabetic mice and suggests that such infiltrates may confer a protective effect on pancreatic beta cells. The balance between the Th1/Th2 cytokines may correlate with protection from T1D. This example also demonstrates that lignosulfonate treatment lowers the concentration of anti-insulin antibodies in both diabetic and non-diabetic mice.

In summary, this example demonstrated that treatment with LS Compound delays the onset of T1D and increases the number of infiltrating CD8+ T cells, expression of IFN-R and IL-10 in NOD mice. Although the mechanisms underlying the effects of LS Compound on cellular infiltration and Beta cell protection need to be investigated further, the current results may facilitate the formulation of preventive-care strategies for T1D.

### Example 7—Administration of Lignosulfonate to Livestock

As indicated in Examples 1 and 4 above, livestock which have been fed lignosulfonate as a supplement to animal feed exhibit excellent health characteristics including resistance to a broad range of pathogenic and medical disorders without the need to administer vaccines, anti-microbial agents, anti-parasitic agents or other veterinary medicines. In particular, in one study swine aged from four months to greater than one year in age were administered lignosulfonate (TemStik) in a dosage within the range of approximately 2-3 g of TemStik per kg of livestock weight. Serological antibody tests were performed on 22 randomized samples from the swine, including tests relating to Mycoplasma hyopneumoniae, swine influenza (H1N1, H3N2), Actinobacillus pleuropneumoniae (APP, 15 serotypes), circovirus type 2 (PCV-II), TGE/PRCV, Porcine Reproductive Respiratory Syndrome (PRRS) and Porcine Enteritis Panel for porcine epidemic diarrhea (PED) TGE Deltal Corona. None of the pigs in the swine herd exhibited symptoms of clinical disease. Serological testing of the swine herd found no circulating viruses even in instances where the herd previously tested positive, for example for H1N1 and H3N2. The absence of the undetected antibodies was somewhat surprising since such antibodies were present in earlier tests of the swine herd, e.g. ancestor animals.

### Example 8—Effect of Lignosulfonate Preparations on Herpes Simplex Virus Type-1 (HSV-1)

The purpose of this example study was to investigate the efficacy of test agent preparations (pC60) comprising lignosulfonate against HSV-1 using a modified plaque reduction assay. The test agent preparation referred to herein as WP consisted of 50% LS and 50% magnesium by volume. The test preparation referred herein as LS consisted of 100% lignosulfonate. The test preparation referred to herein as 0.45 consisted of 55% sulphur and 45% lignosulfonate by volume.

The following materials and methods were used in this study.

### Materials:

1. Virus: HSV-1, Strain F (ATCC VR733)
2. Cell line: African Green Monkey Kidney Epithelial (Vero) cells
3. Growth medium/maintenance medium: Minimal Essential Medium (MEM) with 10% fetal bovine serum in the growth medium
4. Plaque Assay Medium: EMEM (2× w/o phenol red and L-glutamine)
5. Agarose (Low melt; Molecular Biology Grade)
6. Overlay medium (0.8% agarose (w/v) in distilled, deionized water; sterilized in the autoclave prior to use)
7. Dilution solution for pC60: Dulbecco's Phosphate-Buffered Saline (DPBS) for Toxicity Assay; 2×EMEM w/o phenol red and L-glutamine for Plaque Reduction Assay (PRA)
8. Fixitive Solution: 10% buffered Formalin in PBS
9. Staining Solution (PRA): Crystal Violet stain (0.41%)
10. Staining Solution (Toxicity Assay): Trypan Blue solution (4%)
11. Test agent preparations (pC60): WP, LS, and 0.45, as described above.

### Methods:

### Sample Handling

The pC60 test samples were stored at 4° C. Prior to performing each assay, the samples were brought to room temperature and vortexed immediately before preparing dilutions. Virus stocks were stored at −80° C. until the plaque reduction assay was prepared. Virus stock were rapidly thawed and diluted in maintenance medium prior to adding to cells. The virus titer of the stock was determined to be 3.8×10⁶ PFU/mL so a dilution of 104 was utilized for the plaque assays.

### Trypan Blue Cellular Debris (TBCD) Assay

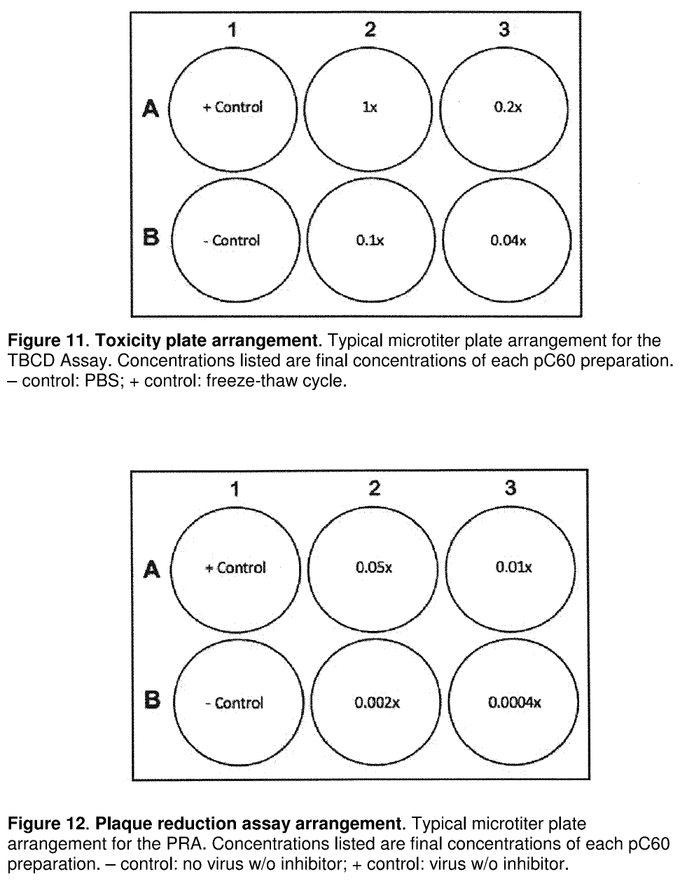

A Trypan Blue Cellular Debris (TBCD) assay was performed. Standard toxicity assays, such as MTT assays, are not ideal for evaluating toxicity with the pC60 family of compounds as they may alter metabolic functions within the cell. Therefore, a novel and validated assay relying on staining cellular debris released from tissue culture, that exhibited comparable results to commercial VITT assays and was not reliant on metabolic measurement was utilized³⁸. Vero cells were seeded into a 6-well plate with 1 mL of growth medium and allowed to grow overnight. Cells were visually inspected prior to infection to confirm the approximate confluency of 80% and normal appearance. Fresh medium was added to each well. Dilutions of each of the pC60 preparations were freshly prepared at 1×, 0.2×, 0.1×, and 0.04× concentrations. Two wells were treated with PBS to serve as a positive (100% cell death after freeze/thaw cycle) and negative control. The plate was arranged according to FIG. 11. Cells were allowed to incubate at 37° C. for 48 hours.

Following incubation, the culture medium was carefully collected to avoid disturbing the monolayer. The plate was then placed at −20° C. for approximately 20 minutes to freeze the positive control and generate a 100% cell death reference. 1 mL of PBS was added to the positive control well and a cell scraper was used to remove the cell monolayer. The cell-containing PBS was collected for analysis. Cellular debris was pelleted via centrifugation for 2 minutes at 10,000×g. The supernatant was carefully removed and 100 μL of Trypan Blue solution was added to each tube, vortexed, and allowed to incubate for 5 minutes at room temperature. The stained sample was then centrifuged at 12,000×g for 2 minutes to pellet the debris and the supernatant containing unreacted dye was removed. The pellet was washed with 500 μl of 99% ethanol and the wash was carefully removed. 100 μL of PBS was added to each pellet, vortexed, and incubated in a heat block for 10 minutes at 80° C. The samples were then centrifuged to pellet debris and the trypan blue PBS extract was collected. Technical duplicates of 45 IAL were added to a 96-well plate and the optical density of the extract was measured by a spectrophotometer at a wavelength of 590 nm.

In addition to the positive and negative controls mentioned above, a background blank control was made that was trypan blue with no debris to measure and account for the residual binding of trypan blue to the centrifuge tubes. Cell death was calculated using the following equation:

Experimental Value−Blank Value

% Cell Death=×100 100% Cell Death Value−Blank Value

### Plaque Reduction Assay (PRA)

A modified plaque reduction assay was performed using the Clinical and Laboratory Standards Institute M33-A standard³⁹. Vero cells were seeded into a 6-well plate with 2 rnL of growth medium and allow to grow overnight at 37° C. Cells were visually inspected prior to infection to confirm the approximate confluency of 100% and normal appearance. Previously titered virus stock was diluted to produce 50 to 100 plaques per 0.2 mL. Each well, except the negative control, received 0.2 mL of diluted virus stock. The negative control received 0.2-mL of fresh maintenance medium. The plate was loaded according to FIG. 12.

An agarose overlay was prepared by mixing overlay medium with diluted pC60 preparations (1:1). The agarose solution was held at 45° C. until after inhibitor dilutions were prepared to keep the agarose in solution. After virus adsorption and removal of unbound virus, 4 mL of overlay was added to the corresponding well and allowed to solidify at room temperature for 30 minutes. The virus was allowed to infect and form plaques for 48 hours while incubating the plate at 37° C. Cells were fixed by adding 4 mL of 10% buffered formalin to each well and incubating for 1 hour at room temperature. Following fixation, the agarose plugs were carefully removed and the staining solution was added to each well. The stain was washed off with water and the plaques were counted for each well.

### Results

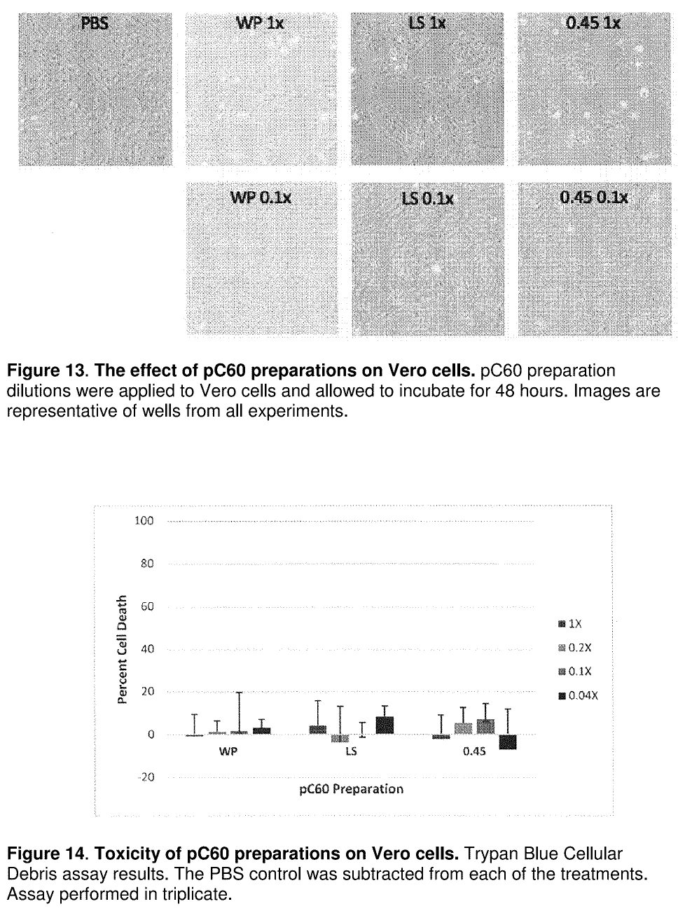

The toxicity of the pc60 compounds were evaluated, qualitatively (FIG. 13) and quantitatively (FIG. 14), on Vero cells at varying concentrations to measure the toxicity and potentially provide additional information regarding mechanism. Due to the hypothesis that the compounds alter cellular metabolism, standard toxicity assays that metabolize tetrazolium salts could not be effectively used. A novel assay that measures the staining of cellular debris present in tissue culture was utilized to provide a quantitative measurement of toxicity. Both the qualitative and quantitative assays resulted in no significant difference in cell death when compared to a PBS control (FIGS. 13 and 14). However, at higher concentrations the growth of the cells was stunted, as demonstrated by large gaps in the cell monolayer, but this effect was diluted out with lower concentrations of compounds. Dilutions of the preparations ultimately resulted in growth comparable to the PBS control (FIG. 13). The WP preparation appeared to result in less inhibition of growth compared to the LS and 0.45 preparations when compared side-by-side at the same dilution. In the TBCD assay, all compounds resulted in cellular death less than 10% after subtraction of the PBS control. Additionally, there were no trends for the different dilutions of each compound.

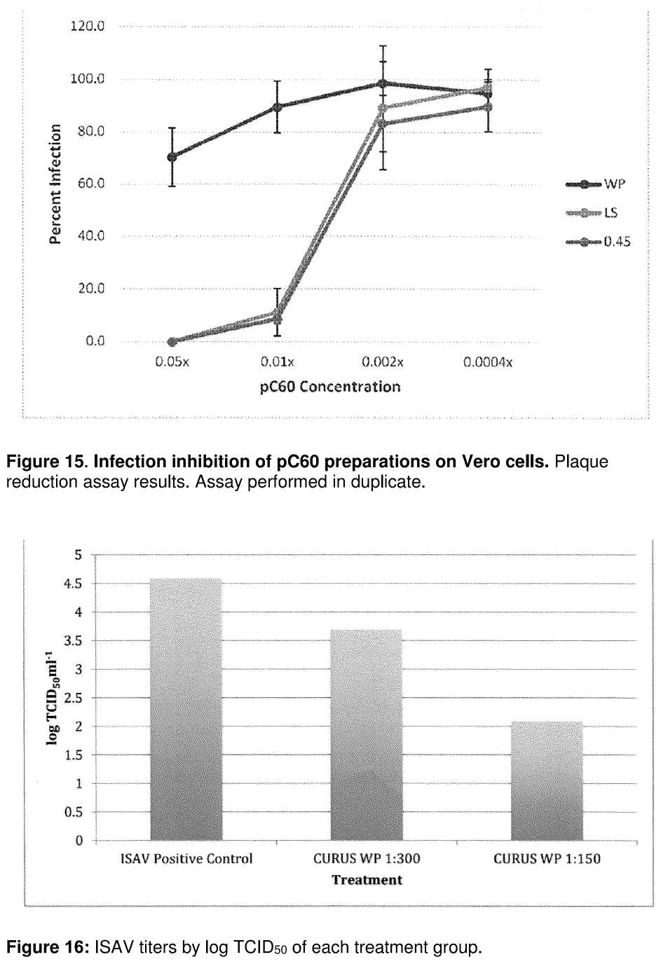

The effectiveness of pC60 preparations on reducing plaques was tested against HSV-1 strain F (VR733). Dilutions of 0.5×, 0.01×, 0.002×, and 0.0004× were chosen based on previous data showing that the 0.05× concentration was able to fully neutralize the virus. Plaque reduction assays were performed in duplicate. The LS and 0.45 preparations displayed more potent HSV-1 infection inhibition than the WP preparation with complete inhibition curves being generated with the former preparations (FIG. 15). Using Prism 9 software, a non-linear regression was performed on the inhibition curve to determine IC50 values for each of the preparations. The less potent WP preparation displayed a calculated IC50 of 0.068× with a R-squared value of 0.7622 since the curve was not complete (Table 4). However, the LP and 0.45 had IC50 values of 0.00575× and 0.00507×, respectively (Table 4). R-squared values were 0.9118 for the LS curve and 0.9370 for 0.45 curve suggesting a good fit between the data.

TABLE 4
Summary of infection inhibition analysis.

| pC60 Preparation | 1050 | R-squared |
|---|---|---|
| WP | 0.06850 | 0.7622 |
| LS | 0.00575 | 0.9118 |
| 0.45 | 0.00507 | 0.9370 |

Prism 9 non-linear regression analysis performed. IC50 values are in times pC60.

### Conclusions

The pC60 preparations were well tolerated even at high concentrations with Vero cells as confirmed by both qualitative (Imaging) and quantitative (TBCD) analyses. Growth appeared slowed at the higher concentrations, but it did not lead to enhanced cell death during the 48 hours of the assay incubation. Cellular death in the assay was very comparable to the PBS control that was performed in tandem, which demonstrates a good safety profile for these compounds with Vero cells. The lack of a dilution trend in the TBCD assays would suggest no direct correlation of cell death due to the preparations.

In the plaque reduction assays, all preparations showed efficacy in inhibiting HSV-1 infection with Vero cells. The LS and 0.45 were roughly an order of magnitude more potent than the WP preparation under the conditions of this study. The LS and 0.45 preparations had IC50 values of approximately 0.005× compared to a calculated IC50 of approximately 0.07× for the WP preparation. This results in roughly 1:150 and 1:2000 dilutions for WP and LS/0.45 preparations, respectively, to obtain a 50% reduction in HSV-1 infection on Vero cells.

The LS and 0.45 preparations had a larger effect with slowing cell growth, which appears to match the antiviral potency.

### Example 9—Effect of Lignosulfonate Preparations on African Swine Fever (ASF) Virus

In this example the anti-viral properties of a test compound designated FR-158 20×p660 (the “Test Compound”) comprising 50% lignosulfonate and 50% elemental sulphur by volume against African Swine Flu (ASF) was investigated. In particular, the toxicity and inhibition of the Test Compound on ASF viral replication on porcine alveolar macrophage (PAM) cell culture was investigated.

### Toxicity Test on PAM Cells Only

The Test Compound was diluted at 1/50, 1/100, 1/200, 1/400 and 1/800 dilutions in AMEM supplemented with Glutamax & Gentamicin+5% Horse Serum.
1. PAM cells on 96-well plate at ˜90% confluency from RDU.
2. Remove media from 96-well plate using multi-channel pipette.
3. Add 200 ul AMEM+5% HS on Columns 1 & 2 row A-H. This will be the cell control.
4. Dilutions of compound added at 200 ul per well in duplicate columns, 8 repeats.
5. Plates incubated at 370+5% 002 for 3 days.

The configuration of the 96-well plate is shown in Table 5 below.

TABLE 5

| | 1 | 2 | 3 | 4 | 5 | 6 | 7 | 8 | 9 | 10 | 11 | 12 |
|---|---|---|---|---|---|---|---|---|---|---|---|---|
| A | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| B | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| C | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| D | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| E | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| F | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| G | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |
| H | CTRL | CTRL | 1/ | 1/ | 1/ | 1/ | 1/ | 1/ | 1/ | 1/ | 1/ | 1/ |
| | | | 50 | 50 | 100 | 100 | 200 | 200 | 400 | 400 | 800 | 800 |

After 3 days of incubation there was a significant change in media colour of the wells treated with Test Compound compared to the cell control. When observed on microscope at 40× magnification, monolayer were still present on entire plate. There seemed to be normal cell division on control wells and wells treated with different dilutions of the Test Compound. No cell toxicity observed.

ASF Virus Treated with Test Compound

ASF virus (Malawi genotype II) was treated with Test Compound diluted at 1/50 final according to the following protocol:
PAM cells seeded at a density of approximately 100,000 cells per well.
ASF Malawi titre—10^{6.5} TCID50/mL.
Stock virus diluted in media to M01-1 then diluted 10-fold to get M01-0.1, M010.01, MOI-0.001.
1. PAM cells on 96-well plate at ˜90% Confluency from RDU.
2. Remove media from 96-well plate using multi-channel pipette.
3. Pre-treat the cells on row A-D column 1-4 by adding 100 uL of 1/50 dilution compound for about 2 hours.
4. Add 200 uL of media on control wells. Add 100 uL of sample dilution series on appropriate wells; row E & F, 1-12.
5. Add 100 uL of media on row E-H 1-12. Add diluted samples on row A-D 1-8 (MOI 1-0.001)

The configuration of the 96-well plate is shown in Table 6 below.

TABLE 6

| | TREATED | | | | UN-TREATED | | | | | | | |
|---|---|---|---|---|---|---|---|---|---|---|---|---|
| | 1 | 2 | 3 | 4 | 5 | 6 | 7 | 8 | 9 | 10 | 11 | 12 |
| A | MOI 1 | MOI 1 | MOI 1 | MOI 1 | MOI 1 | MOI 1 | MOI 1 | MOI 1 | CTRL | CTRL | CTRL | CTRL |
| B | MOI | MOI | MO1 | MOI | MOI | MOI | MOI | MOI | CTRL | CTRL | CTRL | CTRL |
| | 0.1 | 0.1 | 0.1 | 0.1 | 0.1 | 0.1 | 0.1 | 0.1 | | | | |
| C | MOI | MOI | MOI | MOI | MOI | MOI | MOI | MOI | CTRL | CTRL | CTRL | CTRL |
| | 0.01 | 0.01 | 0.01 | 0.01 | 0.01 | 0.01 | 0.01 | 0.01 | | | | |
| D | MOI | MOI | MOI | MOI | MOI | MOI | MOI | MOI | CTRL | CTRL | CTRL | CTRL |
| | 0.001 | 0.001 | 0.001 | 0.001 | 0.001 | 0.001 | 0.001 | 0.001 | | | | |
| E | 10⁰ | 10⁻¹ | 10⁻² | 10⁻³ | 10⁻⁴ | 10⁻⁵ | 10⁻⁶ | 10⁻⁷ | 10⁻⁸ | 10⁻⁹ | 10⁻¹⁰ | 10⁻¹¹ |
| F | 10⁰ | 10⁻¹ | 10⁻² | 10⁻³ | 10⁻⁴ | 10⁻⁵ | 10⁻⁶ | 10⁻⁷ | 10⁻⁸ | 10⁻⁹ | 10⁻¹⁰ | 10⁻¹¹ |
| G | 1/50 | 1/50 | 1/50 | 1/50 | 1/50 | 1/50 | 1/50 | 1/50 | 1/50 | 1/50 | 1/50 | 1/50 |
| H | 1/50 | 1/50 | 1/50 | 1/50 | 1/50 | 1/50 | 1/50 | 1/50 | 1/50 | 1/50 | 1/50 | 1/50 |

After 3 days of incubation, when observed on microscope at 40× magnification, a monolayer was still present on entire plate. There seemed to be normal cell division on control wells and wells treated with different dilutions of Test Compound. No cell toxicity was observed. The plate was kept in ˜70 C freezer. In order to test for anti-viral activity, the plate was thawed and supernatant harvested from treated, un-treated and dilution series wells for nucleic acid extraction. Samples were tested using ASF real time PCR Tignon primers. The test results are set out in Table 7 and Table 8 below.

TABLE 7

| | TREATED | | | | UN-TREATED | | | |
|---|---|---|---|---|---|---|---|---|
| | Well | Well | Well | Well | Well | Well | Well | Well |
| | 1 | 2 | 3 | 4 | 1 | 2 | 3 | 4 |
| MOI | 25.63 | 26.01 | 26.10 | 26.00 | 26.60 | 26.99 | 26.15 | 25.96 |
| 1 | | | | | | | | |
| MOI | 28.42 | 26.99 | 26.34 | 28.46 | 29.21 | 29.74 | 29.95 | 28.69 |
| 0.1 | | | | | | | | |
| MOI | 30.88 | 32.25 | 32.71 | 32.01 | 29.79 | 29.56 | 28.91 | 29.36 |
| 0.01 | | | | | | | | |
| MOI | 34.65 | 36.36 | 36.55 | 35.88 | 32.12 | 31.65 | 31.94 | 34.77 |
| 0.001 | | | | | | | | |

TABLE 8
Dilution series

| | Neat | 10-1 | 10-2 | 10-3 | 10-4 | 10-5 | 10-6 | 10-7 | 10-8 | 10-9 | 10-10 | 10-11 |
|---|---|---|---|---|---|---|---|---|---|---|---|---|
| Replicate 1 | 22.08 | 27.38 | 26.56 | 30.80 | 33.67 | 0 | 0 | 0 | 0 | 0 | 0 | 0 |
| Replicate 2 | 21.84 | 26.58 | 27.33 | 30.63 | 34.30 | 38.86 | 0 | 0 | 0 | 0 | 0 | 0 |

At MOI (multiplicity of infection) 0.01 and 0.001, there seemed to be a ten-fold decrease in viral load between ASF treated with Test Compound and ASF treated with no Test Compound. The cell control had no contamination and no toxicity was observed.

### Example 10—Effect of Lignosulfonate Preparations on Infectious Salmon Anemia Virus (ISAV) Replication in Chinook Salmon Embryo (CHSE) Cells

In this example the anti-viral properties of test compounds, namely CURUS comprising 100% lignosulfonate (LS); CURUS-WP (White Powder) comprising 50% LS and 50% magnesium; and CURUS-SUP comprising 50% LS and 50% elemental sulphur by volume, against ISAV replication in CHSE cells was investigated. In particular, the purpose of the study was to determine the effect of CURUS, CURUS-WP and CURUS-SUP at 1:300 and 1:150 concentrations on ISAV replication in CHSE cells using an end point dilution assay, tissue culture infectious dose 50 (TCID₅₀).

### Cell Culture Preparation

The base medium for this cell line was Minimum Essential Medium (MEM) medium. To make the complete growth medium, 5% fetal bovine serum and 0.0001% gentamycin was added. (Note: The MEM medium formulation was devised for use in a 5% CO2 and air mixture when used for cultivation).

Seven 96 well plates of Chinook Salmon Embryo (CHSE) cells from two 75 cm² flasks of CHSE cells were prepared at a 1:5 ratio allowing for 70-75% cell confluence with seeding. For each 96 well plate, 100 μl of cell suspension was added to each well. Prepared plates were incubated at 16° C. for 24 hours prior to addition of CURUS, CURUS-WP and CURUS-SUP treatments at 1:300 and 1:150 concentrations.

### Preparation of CURUS, CURUS-WP and CURUS-SUP Test Compounds

Stock liquid samples of the test compounds CURUS, CURUS-WP and CURUS-SUP were provided. The stock solutions were held at 4° C. and were vortexed for 15 seconds and allowed to acclimate to room temperature before use.

Test concentrations in MEM cell culture medium were prepared as follows. A total of 16 mls of each test concentration was required but 30 mls was prepared for extra. Please note that media was prepared at double the final concentration of CURUS, CURUS-WP and CURUS-SUP needed so as to achieve the desired final concentration when the serial dilution of ISAV is added to the wells. Essentially a final concentration 1:300 and 1:150 of CURUS, CURUS-WP and CURUS-SUP was used and test concentrations prepared as shown in Table 9 below:

TABLE 9

| | | | MEM Media |
|---|---|---|---|
| | 2x | CURUS | 5% FBS + |
| Formulation | Concentration | (mLs) | gent (mLs) |
| A | Control | 0.0 | MEM Media |
| | | | Only |
| B | 1:150 | .2 | 29.8 |
| | (final 1:300) | | |
| | CURUS | | |
| C | 1:75 | .4 | 29.6 |
| | (final 1:150) | | |
| | CURUS | | |
| | | | MEM Media |
| | 2x | CURUS-WP | 5% FBS + |
| Formulation | Concentration | mLs) | gent (mLs) |
| D | 1:150 | .2 | 29.8 |
| | (final 1:300) | | |
| | CURUS-WP | | |
| E | 1:75 | .4 | 29.6 |
| | (final 1:150) | | |
| | CURUS-WP | | |
| | | | MEM Media |
| | 2x | CURUS-SUP | 5% FBS + |
| Formulation | Concentration | (mLs) | gent (mLs) |
| F | 1:150 | .2 | 29.8 |
| | (final 1:300) | | |
| | CURUS-SUP | | |
| G | 1:75 | .4 | 29.6 |
| | (final 1:150) | | |
| | CURUS-SUP | | |

### ISAV Dilution Preparation

A frozen stock of ISAV was thawed and filtered through a 0.45 μm filter to remove any cellular clumping. A serial dilution from 10⁻¹ out to 10⁻⁸ was then preformed. To ensure that all seven 96 well plates were inoculated with the identical serial dilution, the serial dilution was performed with 9 ml dilution blanks of MEM 5% FBS+gent. The serial dilution tubes were chilled during use and used within 30 minutes of preparation.

### Treatment of 96 Well Plates

Seven 96 well plates were used for the test concentrations of CURUS, CURUS-WP and CURUS-SUP. Two 96 well plates per compound were used and one 96 well plate was used as an ISAV positive control for comparative titer. Plates were labeled with date, cell type, and concentrations. All MEM 5% FBS+gent growth media was aspirated off each of the 96 wells plates.

For each of the test plates, 100 μl of the corresponding test concentration was added to each well of eight rows, up to 10 columns of the 96 well plates. The final two columns were inoculated with control medium only. This allows for 2 control treatment columns for observation of the compound effect on cells and two control columns for observation of the untreated cells. The ISAV positive control plate received control media only. For control wells 200 μl MEM 5% FBS+gent was added to the remaining four columns of this plate.

### ISAV Inoculation of 96 Well Plates

The seven 96 well plates treated with the test concentrations of CURUS, CURUS-WP and CURUS-SUP and the positive control plate were inoculated with 100 μl per well/per serial dilution. Eight wells per dilution per row were inoculated starting with 10-1 and ending with 10⁻⁸. The last four rows were used as cell controls and were not inoculated with ISAV serial dilutions. Each plate was inoculated as presented in Table 10 below.

TABLE 10
ISAV positive control plate(A)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | C | C | C | C |
| 10⁻′ | 10⁻² | 10⁻³ | 10⁻⁴ | 10⁻⁵ | 10⁻⁶ | 10⁻⁷ | 10⁻⁸ | C | C | C | C |
| 10′′′ | 10⁻² | 10⁻³ | 10⁻⁴ | 10⁻⁵ | 10⁻⁶ | 10⁻⁷ | 10⁻⁸ | C | C | C | C |
| 10⁻¹ | 10⁻² | 10⁻³ | 10⁻⁴ | 10⁻⁵ | 10⁻⁶ | 10⁻⁷ | 10⁻⁸ | C | C | C | C |
| 10⁻¹ | 10⁻² | 10⁻³ | 10⁻⁴ | 10⁻⁵ | 10⁻⁶ | 10⁻⁷ | 10⁻⁸ | C | C | C | C |
| 10⁻¹ | 10⁻² | 10⁻³ | 10⁻⁴ | 10⁻⁵ | 10⁻⁶ | 10⁻⁷ | 10⁻⁸ | C | C | C | C |
| 10⁻¹ | 10⁻² | 10⁻³ | 10⁻⁴ | 10⁻⁵ | 10⁻⁶ | 10⁻⁷ | 10⁻⁸ | C | C | C | C |
| 10⁻¹ | 10⁻² | 10⁻³ | 10⁻⁴ | 10⁻⁵ | 10⁻⁶ | 10⁻⁷ | 10⁻⁸ | C | C | C | C |

CURUS 1:300(B)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻′ | 10⁻² | 10⁻³ | 10⁻⁴ | 10⁻⁵ | 10⁻⁶ | 10⁻⁷ | 10⁻⁸ | 1:300 | 1:300 | C | C |
| 10′′′ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |

CURUS 1:150 (C)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻′ | 10⁻² | 10⁻³ | 10⁻⁴ | 10⁻⁵ | 10⁻⁶ | 10⁻⁷ | 10⁻⁸ | 1:150 | 1:150 | C | C |
| 10′′′ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |

CURUS-WP 1:300 (D)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻′ | 10⁻² | 10⁻³ | 10⁻⁴ | 10⁻⁵ | 10⁻⁶ | 10⁻⁷ | 10⁻⁸ | 1:300 | 1:300 | C | C |
| 10′′′ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |

CURUS-WP 1:150 (E)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻′ | 10⁻² | 10⁻³ | 10⁻⁴ | 10⁻⁵ | 10⁻⁶ | 10⁻⁷ | 10⁻⁸ | 1:150 | 1:150 | C | C |
| 10′′′ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |

CURUS-S UP 1:300 (F)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻′ | 10⁻² | 10⁻³ | 10⁻⁴ | 10⁻⁵ | 10⁻⁶ | 10⁻⁷ | 10⁻⁸ | 1:300 | 1:300 | C | C |
| 10′′′ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |
| 10⁻¹ | 10⁻² | 10⁻³ | 10⁻⁴ | 10⁻⁵ | 10⁻⁶ | 10⁻⁷ | 10⁻⁸ | 1:300 | 1:300 | C | C |

CURUS-S UP 1:150(G)

| | | | | | | | | | | | |
|---|---|---|---|---|---|---|---|---|---|---|---|
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻′ | 10⁻² | 10⁻³ | 10⁻⁴ | 10⁻⁵ | 10⁻⁶ | 10⁻⁷ | 10⁻⁸ | 1:150 | 1:150 | C | C |
| 10′′′ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |
| 10⁻¹ | 10⁻² | 10⁻³ | 10⁻⁴ | 10⁻⁵ | 10⁻⁶ | 10⁻⁷ | 10⁻⁸ | 1:150 | 1:150 | C | C |

### Results

ISAV titers were determined by end point dilution assay calculations, TCID₅₀. Infected cells treated with CURUS and CURUS SUP dilutions at 1:300 and 1:150 are shown in Table 11 below.

TABLE 11

| | Log | | Cumulative | Cumulative | Ratio | | | |
|---|---|---|---|---|---|---|---|---|
| | Virus | | infected | uninfected | A/ | % | Proportionate | log |
| Sample | dilution | Infected | A | B | (A + B) | Infected | distance | TCID50 |
| ISAV Control | −2 | 8/8 | 17 | 0 | 1.00 | 100.0 | | |
| | −3 | 7/8 | 9 | 1 | 0.90 | 90.0 | 0.6 | 4.6 |
| | −4 | 2/8 | 2 | 7 | 0.22 | 22.2 | | |
| | −5 | 0/8 | 0 | 15 | 0.00 | 0.0 | | |
| CURUS WP | −1 | 8/8 | 10 | 0 | 1.00 | 100.0 | 0.7 | 3.7 |
| 1:300 | | | | | | | | |
| | −2 | 2/8 | 2 | 6 | 0.25 | 25.0 | | |
| | −3 | 0/8 | 0 | 14 | 0.00 | 0.0 | | |
| CURUS WP | 0 | 8/8 | 20 | 0 | 1.00 | 100.0 | | |
| 1:150 | | | | | | | | |
| | −1 | 7/8 | 12 | 1 | 0.92 | 92.3 | | |
| | −2 | 5/8 | 5 | 4 | 0.56 | 55.6 | 0.1 | 2.1 |
| | −3 | 0/8 | 0 | 12 | 0.00 | 0.0 | | |

ISAV Positive Control = 1 × 10^{4.6} TCID₅₀ mL⁻¹
CURUS WP 1:300 = 1 × 10^{3.7} TCID₅₀ mL⁻¹
CURUS WP 1:150 = 1 × 10^{2.1} TCID₅₀ mL⁻¹

### Conclusions

As each titer assay for the compounds and dosages tested had only one replicate, there was only one value and therefore no variance for which to run statistics.

With reference to FIG. 16, the virus titers obtained for the CURUS WP treatment indicated there was a 2.5 log difference in the 1:150 dilution when compared to the ISAV Positive Control. Having a greater than 2-log difference may demonstrate a significant difference. Conversely, the virus titers obtained for the CURUS WP treatment at a 1:300 dilution indicated there was a 0.9 log difference when compared to the ISAV Positive Control. Having less than a 2-log difference may demonstrate no significant difference between the CURUS WP at 1:300 dilution and the ISAV Positive Control.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

1. Barnard D L, Heaton K W. Bile acids and vitamin A absorption in man: the effects of two bile acid-binding agents, cholestyramine and lignin. Gut. 1973 April; 14(4):316-8.
2. Myrvold B O. A new model for the structure of lignosulphonates. Part 1. Behaviour in dilute solutions. Industrial Crops and Products. 2008 March; 27(2):214-9.
3. Reddy B S, Maeura Y, Wayman M, DiBello J, Berke B, Simi B, et al. Effect of Dietary Corn Bran and Autohydrolyzed Lignin on 3,2′-Dimethyl-4-aminobiphenyl-Induced Intestinal Carcinogenesis in Male F344 Rats [Internet]. Vol. 71, JNCI. 1983. Available from: https://academic.oup.com/jnci/article/71/2/419/907089
4. Zakzeski J, Bruijnincx P C A, Jongerius A L, Weckhuysen B M. The catalytic valorization of lignin for the production of renewable chemicals. Chem Rev. 2010 Jun. 9; 110(6):3552-99.
5. Reddy B S, Maeura Y, Wayman M. Effect of dietary corn bran and autohydrolyzed lignin on 3,2′-dimethyl-4-aminobiphenyl-induced intestinal carcinogenesis in male F344 rats. J Natl Cancer Inst. 1983 August; 71(2):419-23.
6. Hasegawa Y, Kadota Y, Hasegawa C, Kawaminami S. Lignosulfonic Acid-Induced Inhibition of Intestinal Glucose Absorption. J Nutr Sci Vitaminol (Tokyo). 2015; 61(6):449-54.
7. Hasegawa Y, Nakagawa E, Kadota Y, Kawaminami S. Lignosulfonic acid promotes hypertrophy in 3T3-L1 cells without increasing lipid content and increases their 2-deoxyglucose uptake. Asian-Australas J Anim Sci. 2017 January; 30(1):111-8.
8. Akkati S, Sam K G, Tungha G. Emergence of promising therapies in diabetes mellitus.

J Clin Pharmacol. 2011 June; 51(6):796-804.
9. Reddy V D, Padmavathi P, Paramahamsa M, Varadacharyulu N C. Amelioration of alcohol-induced oxidative stress by Emblica officinalis (amla) in rats. Indian J Biochem Biophys. 2010 February; 47(1):20-5.
10. de Sousa E, Zanatta L, Seifriz I, Creczynski-Pasa T B, Pizzolatti M G, Szpoganicz B, et al. Hypoglycemic effect and antioxidant potential of kaempferol-3,7-O-(alpha)-dirhamnoside from Bauhinia forficata leaves. J Nat Prod. 2004 May; 67(5):829-32.
11. Bailey C J. Metformin: historical overview. Diabetologia. 2017; 60(9):1566-76.
12. Rajadurai M, Prince P S M. Preventive effect of naringin on isoproterenol-induced cardiotoxicity in Wistar rats: an in vivo and in vitro study. Toxicology. 2007 Apr. 11; 232(3):216-25.
13. Thompson J L, Mallet-Boucher M, McCloskey C, Tamlyn K, Wilson K. Educating nurses for the twenty-first century abilities-based outcomes and assessing student learning in the context of democratic professionalism. Int J Nurs Educ Scholarsh. 2013 Oct. 9; 10.
14. Barnard D L, Heaton K W. Bile acids and vitamin A absorption in man: the effects of two bile acid-binding agents, cholestyramine and lignin. Gut. 1973 April; 14(4):316-8.
15. Karlsson M G, Lawesson S S, Ludvigsson J. Th1-like dominance in high-risk first-degree relatives of type I diabetic patients. Diabetologia. 2000 June; 43(6):742-9.
16. Halminen M, Simell O, Knip M, Ilonen J. Cytokine expression in unstimulated PBMC of children with type 1 diabetes and subjects positive for diabetes-associated autoantibodies. Scand J Immunol. 2001 May; 53(5):510-3.
17. Hedman M, Ludvigsson J, Faresjö MK. Nicotinamide reduces high secretion of IFN-gamma in high-risk relatives even though it does not prevent type 1 diabetes. J Interferon Cytokine Res. 2006 April; 26(4):207-13.
18. Karlsson Faresjö MGE, Ernerudh J, Ludvigsson J. Cytokine profile in children during the first 3 months after the diagnosis of type 1 diabetes. Scand J Immunol. 2004 May; 59(5):5 17-26.
19. Ryden A, Stechova K, Durilova M, Faresjö M. Switch from a dominant Th1-associated immune profile during the pre-diabetic phase in favour of a temporary increase of a Th3-associated and inflammatory immune profile at the onset of type 1 diabetes.

Diabetes Metab Res Rev. 2009 May; 25(4):335-43.
20. Stechova K, Bohmova K, Vrabelova Z, Sepa A, Stadlerova G, Zacharovova K, et al. High T-helper-1 cytokines but low T-helper-3 cytokines, inflammatory cytokines and chemokines in children with high risk of developing type 1 diabetes. Diabetes Metab Res Rev. 2007 September; 23(6):462-71.
21. Hyöty H, Hiltunen M, Knip M, Laakkonen M, Vahasalo P, Karjalainen J, et al. A prospective study of the role of coxsackie B and other enterovirus infections in the pathogenesis of IDDM. Childhood Diabetes in Finland (DiMe) Study Group. Diabetes. 1995 June; 44(6):652-7.
22. Elfaitouri A, Berg A K, Frisk G, Yin H, Tuvemo T, Blomberg J. Recent enterovirus infection in type 1 diabetes: evidence with a novel IgM method. J Med Virol. 2007 December; 79(12):1861-7.
23. Palmer J P, Asplin C M, Clemons P, Lyen K, Tatpati O, Raghu P K, et al. Insulin antibodies in insulin-dependent diabetics before insulin treatment. Science. 1983 Dec. 23; 222(4630):1337-9.
24. Rich S S. Mapping genes in diabetes. Genetic epidemiological perspective. Diabetes. 1990 November; 39(11):1315-9.
25. Tuomilehto J, Podar T, Tuomilehto-Wolf E, Virtala E. Evidence for importance of gender and birth cohort for risk of IDDM in offspring of IDDM parents. Diabetologia. 1995 August; 38(8):975-82.
26. Bottazzo G F, Florin-Christensen A, Doniach D. Islet-cell antibodies in diabetes mellitus with autoimmune polyendocrine deficiencies. Lancet. 1974 Nov. 30; 2(7892):1279-83.
27. Eisenbarth G S. Update in type 1 diabetes. J Clin Endocrinol Metab. 2007 July; 92(7):2403-7.
28. Eisenbarth G S, Jeffrey J. The natural history of type 1A diabetes. Arq Bras Endocrinol Metabol. 2008 March; 52(2):146-55.
29. Orban T, Sosenko J M, Cuthbertson D, Krischer J P, Skyler J S, Jackson R, et al. Pancreatic islet autoantibodies as predictors of type 1 diabetes in the Diabetes Prevention Trial-Type 1. Diabetes Care. 2009 December; 32(12):2269-74.
30. Soberanes S, Misharin A v, Jairaman A, Morales-Nebreda L, McQuattie-Pimentel A C, Cho T, et al. Metformin Targets Mitochondrial Electron Transport to Reduce Air-Pollution-Induced Thrombosis. Cell Metab. 2019; 29(2):335-347.e5.
31. Rena G, Hardie D G, Pearson E R. The mechanisms of action of metformin.

Diabetologia. 2017; 60(9):1577-85.
32. Arif S, Leete P, Nguyen V, Marks K, Nor N M, Estorninho M, et al. Blood and islet phenotypes indicate immunological heterogeneity in type 1 diabetes. Diabetes. 2014 November; 63(11):3835-45.
33. Kefas B A, Cai Y, Kerckhofs K, Ling Z, Martens G, Heimberg H, et al. Metformin-induced stimulation of AMP-activated protein kinase in beta-cells impairs their glucose responsiveness and can lead to apoptosis. Biochem Pharmacol. 2004 Aug. 1; 68(3):409-16.
34. Corkey B E. Banting lecture 2011: hyperinsulinemia: cause or consequence? Diabetes. 2012 January; 61(1):4-13.
35. Czech M P. Insulin action and resistance in obesity and type 2 diabetes. Nat Med. 2017 Jul. 11; 23(7):804-14.
36. Ye J. Role of insulin in the pathogenesis of free fatty acid-induced insulin resistance in skeletal muscle. Endocr Metab Immune Disord Drug Targets. 2007 March; 7(1):65-74.
37. Jedrzejczak P, Collins M N, Jesionowski T, Klapiszewski L. The role of lignin and lignin-based materials in sustainable construction—A comprehensive review. Int J Biol Macromol. 2021 Sep. 30; 187:624-50
38. Lebeau, P. F.; Chen, J.; Byun, J. H.; Platko, K.; Austin, R. C. The trypan blue cellular debris assay: a novel low-cost method for rapid quantification of cell death. MethodsX. 2019. 6(2019), 1174-1180. DOI: 10.1016/j.mex.2019.05.010
39. CLSI. Antiviral Susceptibility Testing: Herpes Simplex Virus by Plaque Reduction Assay; Approved Standard. CLSI document M33-A. Wayne, PA: Clinical and Laboratory Standards Institute, 2004.

1. The use of a composition comprising lignosulfonate for the prevention and treatment of pathogenic and medical disorders in humans and animals, wherein said composition is substantially free of elemental sulphur.

2. The use as defined in claim 1, wherein said lignosulfonate is radically polymerized lignosulfonate.

3. The use as defined in claim 1, wherein said composition comprises lignosulfonate selected from the group of ammonium lignosulfonate, sodium lignosulfonate, calcium lignosulfonate and magnesium lignosulfonate.

4. The use as defined in claim 1, wherein said composition is formulated for use as an animal feed additive or supplement.

5. The use as defined in claim 4, wherein said composition is administered in animal feed at a daily dosage of approximately 2-3 g per kg weight of said animal.

6. The use as defined in claim 1, wherein said composition sufficiently attenuates a pathogenic effect of a pathogen present in a human or animal when administered in an effective amount to said human or animal in vivo to enable said human or animal to mount an effective immune or other biological response to said pathogen.

7. The use as defined in claim 1, wherein said pathogenic and medical disorders are selected from the group consisting of disorders caused by microbial pathogens; disorders caused by viral pathogens; disorders caused by prions; disorders caused by protists; disorders caused by fungi; disorders caused by parasites; lung and airway disorders; bone, joint and muscle disorders; digestive disorders; hormonal disorders; cancer; auto immune disorders; neurodegenerative disorders; skin disorders; and sexual and reproductive disorders.

8. The use as defined in claim 7, wherein said pathogenic and medical disorders comprise diabetes mellitus.

9. The use as defined in claim 8, wherein said diabetes mellitus is Type I diabetes.

10. The use as defined in claim 5, wherein said daily dosage is approximately 2.5 g per kg weight of animal.

11. The use as defined in claim 1, wherein said composition is formulated for oral or injectable administration.

12. A method of preventing or treating a pathogenic or medical disorder in a human or animal subject comprising administering to said subject an effective amount of a composition comprising lignosulfonate, wherein said composition is substantially free of elemental sulphur.

13. The method as defined in claim 12, wherein said lignosulfonate is radically polymerized lignosulfonate.

14. The method as defined in claim 12, wherein said composition comprises lignosulfonate selected from the group of ammonium lignosulfonate, sodium lignosulfonate, calcium lignosulfonate and magnesium lignosulfonate.

15. The method as defined in claim 12, wherein said composition is formulated for administration as an animal feed additive.

16. The method as defined in claim 15, wherein said composition is administered at a daily dosage of approximately 2-3 g per kg weight of said animal.

17. The method as defined in claim 12, wherein said composition sufficiently attenuates a pathogenic effect of a pathogen present in said subject to enable said subject to mount an effective immune or other biological response to said pathogen.

18. The method as defined in claim 12, wherein pathogenic and medical disorders are selected from the group consisting of disorders caused by microbial pathogens; disorders caused by viral pathogens; disorders caused by prions; disorders caused by protists; disorders caused by fungi; disorders caused by parasites; lung and airway disorders; bone, joint and muscle disorders; digestive disorders; hormonal disorders; cancer; auto immune disorders; neurodegenerative disorders; skin disorders; and sexual and reproductive disorders.

19. The method as defined in claim 18, wherein said pathogenic and medical disorders comprise diabetes mellitus.

20. The method as defined in claim 19, wherein diabetes mellitus is Type I diabetes.

21. The method as defined in claim 16, wherein said daily dosage is approximately 2.5 g per kg weight of animal.

22. The method as defined in claim 12, wherein said administration is by oral administration.

23. The method as defined in claim 12, wherein said administration is by intraperitoneal injection.

24. A pharmaceutic composition comprising lignosulfonate, wherein said composition is substantially free of elemental sulphur and is formulated in a dosage for use in the prevention or treatment of a pathogenic or medical disorder in a human or animal subject.

25. The composition as defined in claim 24, wherein said composition is formulated in a daily dosage of approximately 2-3 g per kg weight of said animal.

26. The composition as defined in claim 25, wherein said daily dosage is approximately 2.5 g per kg weight of said animal.

27. The composition as defined in claim 24, wherein lignosulfonate is radically polymerized lignosulfonate.

28. The composition as defined in claim 24, wherein said animal is a swine.

29. The use of a composition comprising lignosulfonate for the prevention and treatment of Type 1 diabetes in humans and animals.

30. The use as defined in claim 29, wherein said lignosulfonate is radically polymerized lignosulfonate.

31. The use as defined in claim 29, wherein said composition comprises lignosulfonate selected from the group of ammonium lignosulfonate, sodium lignosulfonate, calcium lignosulfonate and magnesium lignosulfonate.

32. The use as defined in claim 29, wherein the composition alters the balance of serum concentration of Th1/Th2 cytokines as compared to untreated subjects.

33. The use as defined in claim 29, wherein the composition attenuates the concentration of anti-insulin antibodies in the serum.

34. The use as defined in claim 29, wherein the composition increase the serum concentration of one or more cytokines selected from the group consisting of TNF-α IFN-γ, IL-6, and IL-10.

35. The use as defined in claim 29, wherein the composition increases the pancreatic infiltration of CD8+ T cells.

36. A method of preventing or treating Type 1 diabetes in a human or animal subject comprising administering to said subject an effective amount of a composition comprising lignosulfonate.

37. A composition comprising lignosulfonate formulated for treatment of Type 1 diabetes in a human or animal subject.
